(19) United States
(12) Patent Application Publication
(10) Pub. No.: US 2024/0374214 A1
(43) Pub. Date: Nov. 14, 2024

(54) IMPLANTABLE AND CONTINUOUS MULTI-ANALYTE MONITOR (MAM) SENSOR

(71) Applicant: THE REGENTS OF THE UNIVERSITY OF CALIFORNIA, Oakland, CA (US)

(72) Inventors: Elliot L. Botvinick, Irvine, CA (US); John Weidling, Irvine, CA (US); Mark Keating, Irvine, CA (US); Dat Nguyen, Irvine, CA (US); Micah Malik Lawrence, Irvine, CA (US)

(21) Appl. No.: 18/690,587

(22) PCT Filed: Sep. 7, 2022
(86) PCT No.: PCT/US22/76010

§ 371 Date: Mar. 8, 2024

Related U.S. Application Data
(60) Provisional application No. 63/242,386, filed on Sep. 9, 2021.

(51) Int. Cl. A61B 5/00; A61B 5/145

(52) U.S. Cl. CPC A61B 5/686; A61B 5/14539; A61B 5/14546

(57) ABSTRACT

Disclosed herein are embodiments of a continuous multi-analyte monitor that can be used to measure one or more analytes, for example pH and lactate, in a patient. In some embodiments, the sensor can be implanted in the tissue of a patient, and a recording unit can be located on the skin of the patient, generally adjacent to the sensor. Detectors located on the sensor and on the recording unit can detect luminescent signals that originate from the implantable sensor and can be used to determine analyte concentrations, or otherwise relevant values.

### CROSS-REFERENCES TO RELATED APPLICATIONS

This application claims benefit of U.S. Provisional Application No. 63/242,386 filed Sep. 9, 2021, the specification(s) of which is/are incorporated herein in their entirety by reference.

### STATEMENT REGARDING FEDERALLY SPONSORED RESEARCH OR DEVELOPMENT

This invention was made with government support under Grant Nos. T32HL 116270 and T34GM069337, awarded by the National Institutes of Health (NIH). The government has certain rights in the invention.

### FIELD OF THE INVENTION

The disclosure relates generally to monitoring analytes in a patient using an implantable and continuous multi-analyte monitor (MAM), comprising an implantable flexible sensor and recording unit, in order to monitor the health of a patient.

### BACKGROUND OF THE INVENTION

In diseases such as sepsis and septic shock, pH and lactate have been considered as vital biomarkers towards patient outcome. Regulating pH and lactate levels towards statistically significant changes before and after treatment has generally led to an increased likelihood of patient survival and earlier hospital discharge. Fluctuations in either pH or lactate can be signs of metabolic or respiratory ailments. For example, during inadequate blood oxygenation (hypoxemia), metabolism is directed towards anaerobic glycolysis which excessively produces lactate (hyperlactatemia). Lactate, the conjugate base of a strong acid, yields an equimolar production of hydronium ions (H+) that upon exceeding the bicarbonate buffering system, decreases blood pH making the blood acidic. Unfortunately, fluctuations in pH and lactate are seen during traumatic injury, inhalation of toxic chemical agents, and during diseased metabolic states which, if left untreated, can lead to death.

An often-referenced example for continuously monitoring pH and lactate is sepsis. Approximately, 1.7 million adult Americans develop sepsis each year. Of this population, approximately 270,000 passed away. In 2016, the Healthcare Cost and Utilization Project (HCUP) and the Agency for Healthcare Research and Quality stated that sepsis was responsible for almost $24 billion in annual costs. If left untreated, sepsis can advance to septic shock, which has an even poorer patient outcome. Literature supports that with careful treatment guided by pH and lactate, those suffering with sepsis can have a successful recovery. In a prospective study with 75 patients experiencing sepsis and metabolic acidosis, the 11 non-survivors had a lower mean blood pH and a higher mean blood lactate level. Changes in pH and lactate were not statistically significant (p>0.714) as compared to initial values, whereas significant changes were observed for the 64 survivors (P<0.002). In a similar study where 35 patients also experience sepsis, but gastric intramucosal pH was studied rather than blood pH, lactate concentrations remained high in non-survivors but progressively decreased in survivors, (4 hrs: 3.3+/−1.1 mEq/L in non-survivors vs. 2.2+/−0.9 mEq/L in survivors [p<0.01]; 24 hrs: 3.5+/−2.0 mEq/L in non-survivors vs. 1.9+/−1.1 mEq/L in survivors [p<0.05]). Meanwhile, intramucosal pH was lower in the non-survivors than in the survivors initially (7.19+/−0.15 in non-survivors vs. 7.30+/−0.14 in survivors [p<0.05]), at 4 hrs (7.18+/−0.17 in non-survivors vs. 7.29+/−0.13 in survivors [p=0.06]), and at 24 hrs (7.19+/−0.31 in non-survivors vs. 7.30+/−0.17 in survivors [p<0.05]). Of the 14 patients with persistently high lactate concentrations at 24 hrs, all nine (100%) patients with low intramucosal pH, but only two (40%) of five patients with normal intramucosal pH died (p<0.001).

Current clinical standards for measuring blood pH and lactate require blood draws and analysis by benchtop instrumentations (blood gas analyzer, Yellow Spring Instrument (YSI) lactate analyzers) or hand-held monitors (i-STAT). The frequency of measurements is limited ultimately by the frequency of blood draws which increase the likelihood of blood infections, nerve damage, hematoma, and anemia. In a retrospective study determining the effect of phlebotomy on hemoglobin and hematocrit, every 100 ml of phlebotomy was associated with a reduction of 7.0 g/L and 19% in hemoglobin and hematocrit, respectively. In intensive care situations, such as cardiovascular surgery after myocardial infarction, where the patient is already exhibiting a high degree of stress, the reduction in red blood cells and hematocrit has been cited to increase the likelihood of morbidity. In a review of 19,617 radial artery cannulations (where blood is drawn to measure blood gas), temporary occlusion of the artery was observed in 19.70% of those cases. Studies show that such arterial occlusions may lead to local ischemia and stroke, with poorer clinical outcomes associated with older aged patients. Needle insertion into the subcutaneous space also carries the risk of nerve damage. The most common nerve damage involves the lateral antebrachial cutaneous nerve which can lead to complex regional pain syndrome. The incidence of nerve injury was found to be between 1 in 21,000 and 1 in 26,000. Although chronic ailments associated with venipuncture associated nerve damage are rare (1 in 1.5 million phlebotomies), 87% of these patients require care by a pain management specialist to rectify the pain. Though hematomas are rare after arterial blood draws and phlebotomy (<3%), ecchymosis occurs more often (15%) after phlebotomy with a noticeable diameter<20 mm occurring in 90% of cases. Interestingly, hematomas were exhibited at the site of phlebotomy in 12.3% of blood donors, an observed increase likely due to prolonged blood draws at the venipuncture site. A side effect of hematoma is the compression of surrounding nerves that has been noted to occur in 24% of patients.

There is no commercial continuous pH and lactate sensor, but clearly, there is a clinical need and market for it. The state of the art for measuring both analytes is limited to benchtop and handheld analyzers that require invasive blood draws and exhibit long processing time. Although there are handheld lactate sensors that provide measurements within a minute, an increase in lactate can result in a decrease in pH, but this is not always the case. For this reason, it is important to recognize that measuring only lactate or pH is not a surrogate for measuring the other, and that measuring both pH and lactate can provide healthcare professionals more data to improve patient prognosis. For example, during sepsis, blood pH decreases while lactate levels increase. However, during chronic obstructive pulmonary disease, pH decreases but lactate levels do not significantly change. In these conditions, measuring either pH or lactate alone would not provide a sufficient diagnosis and direct the healthcare professional towards an improper mode of treatment. The information provided by a pH and lactate MAM would allow healthcare professionals to gather more clinical data, providing a clearer clinical picture of the ailments affecting the patient. This would allow the healthcare professional to provide precision medicine to help their patient.

### BRIEF SUMMARY OF THE INVENTION

It is an objective of the present invention to provide systems, devices, and methods that allow for detection and monitoring of multiple analytes, as specified in the independent claims. Embodiments of the invention are given in the dependent claims. Embodiments of the present invention can be freely combined with each other if they are not mutually exclusive.

Disclosed herein are embodiments for an implantable and continuous multi-analyte monitor (MAM) comprising, but not limited to, at least two sensing modalities to measure multiple analytes using opto-electronic components, as seen in FIG. 1. In some aspects, the MAM comprises two main components. The first main component is a flexible sensor which may be of non-limiting dimensions less than about 0.01 mm to 50 mm in width, and less than about 0.1 mm to 200 mm in length. The flexible sensor can contain opto-electronic components which may be of non-limiting dimensions 0.001-50 mm in width and 0.001-50 mm in length. The opto-electronic components, such as light sources and detectors, can be inserted into a tissue or tissue compartment. The sensor can comprise a multitude of luminescent probes to sense a plurality of analytes.

In some embodiments, the flexible sensor can have a width of less than 50 microns to 5,000 microns but these dimensions are not limiting. In some embodiments, the flexible sensor can have a length of less than 0.01 cm to 20 cm but these dimensions are not limiting.

In some embodiments, the light source(s) and the photodetector(s) on the flexible sensor can have a length and/or width of less than 50 microns to 1,000 microns but these dimensions are not limiting. In some embodiments, the light source(s) and the photodetector(s) on the flexible sensor can have heights of less than 10 microns to 1000 microns but these dimensions are not limiting.

The second main component is a recording unit that can receive and record signals from the flexible sensor. In some embodiments, the recording unit can have a length and/or width of less than 2 cm to 10 cm but these dimensions are not limiting. In one non-limiting embodiment, the sensor can be placed under the skin and the recording unit can be worn on the skin above the sensor. The flexible sensor can operate by utilizing a plurality of luminescence properties or optical phenomena to detect multiple analytes.

In one sensing modality that is non-limiting, scattered light originating from light sources on the flexible sensor is detected by a photodetector located on the flexible sensor. In another sensing modality that is not limiting, transmitted light originating from the flexible sensor is detected by a photodetector located within a recording unit, such that the recording unit and flexible sensor are separated by tissue, such as that belonging to skin or an internal organ. In some embodiments, the photodiode located in the recording unit can have a length and/or width of less than 0.01 cm to 10 cm but these dimensions are not limiting.

One feature of the flexible sensor is that it contains wires, or electrically conductive traces that conduct electricity to and from the recording unit, and to one or more opto-electronic components on the flexible sensor. As well, the traces can be used to transmit current from photodetector(s) to the recording unit. These wires or electrically conductive traces contain at least one electrically conductive pad on which elements or optoelectronic components can be attached or electrical connections can be made. The wires or electrically conductive traces also contain at least one electrically conductive pad for making electrical connections to other elements including, but not limited to a recording unit, control board, electronic microchip, data acquisition system, or electrically conductive elements such as wires, flexible circuits, and the like. These opto-electronic components can be, but are not limited to, light sources and current sources, such as photodetectors. The flexible sensor can be designed to share wires or electrically conducting traces to turn on the light source(s) and operate/receive signals from the current source, which can reduce the total number of wires/traces required. A benefit of this strategy is a reduction in the overall dimensions of the flexible sensor, which is of importance considering that the flexible circuit can be introduced into a tissue.

Without wishing to be bound by a particular theory or mechanism, the present invention allows for light sources to be activated one at a time thus limiting any optical signal cross talk between each of those light sources and any optical signal crosstalk by chemistries or signaling molecules that may alter the light as part of the sensing strategy. The strategy also allows for two or more sources to be activated or read from simultaneously. As well, this invention allows electrical traces used to receive current from a current source to also be used for light source excitation.

In one embodiment, some or all of the light sources are located on the same plane as the photodetector(s) of the flexible circuit, as shown in FIGS. 1, 3, and 4. In another embodiment, some or all of the light sources are located on the opposite plane as the photodetector(s) of the flexible circuit, as shown in FIGS. 5 and 6. In some embodiments, the light sources and photodetector are arranged either in series along the long axis of the flexible sensor or side-by-side but not along the long axis of the flexible sensor, or some combination of these two arrangements.

In some embodiments, the recording unit contains internal circuitry for activating or supplying power to the sensor's light sources and for receiving and recording luminescence signals. The recording unit can comprise electronic components to digitize an analog signal, including but not limited to data acquisition chips or circuits, microcontrollers, batteries, resistors, inductors, capacitors, operational amplifiers, field effect transistors, switches, fuses, and wireless transmitters/receivers. In one embodiment of the recording unit, the recording unit is situated in a housing unit to prevent damage due to water, biological fluid or interference from external light, electrical fields, mechanical stresses, and other sources of damage or interference. The recording unit can comprise either a rigid or a flexible printed circuit board or other related technologies. In some embodiments the recording unit can comprise a flexible printed circuit that is continuous with the flexible sensor. In some embodiments, the recording unit is connected to a computer with wires and cables to receive and transmit commands and data. In other embodiments, the recording unit is connected to a computer or other device through a wireless protocol such as that from the non-limiting list of Bluetooth, Bluetooth Low Energy (BLE), ZigBee, Wi-Fi, and other radio-frequency or optical protocols. These protocols may be used to receive and transmit commands and data, and may be powered via battery or other non-wired power source. In some embodiments, a combination of diverse connections is used to transmit and receive commands and data.

In some embodiments, the flexible sensors described herein can be used in a method of detecting and monitoring levels of one or more analytes in a subject. The method may comprise providing a MAM system, and implanting at least the sensor unit of the MAM system into the subject. The one or more analytes may be pH, lactate, glucose, ketones, or a combination thereof.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 illustrates a specific but not limiting embodiment of a multi-analyte monitor, with multiple optoelectronic components attached to the flexible sensor inside tissue.

FIG. 2 illustrates a specific but not limiting embodiment of a flexible sensor with electrically conductive pads and wire traces for electronic components, as well as electrically conductive pads for connections to other devices.

FIG. 3 illustrates a specific but not limiting embodiment of a multi-analyte monitor, utilizing two light sources, one photodetector and a sensing sheet for sensing of a first analyte. The embodiment also utilizes two light sources, working and reference sensing sheets, and a photodetector for sensing a second analyte.

FIG. 4 illustrates a specific but not limiting embodiment of a multi-analyte monitor, utilizing a single light source, two photodetectors and a sensing sheet for sensing of a first analyte. The embodiment also utilizes two light sources, working and reference sensing sheets, and a photodetector for sensing a second analyte.

FIG. 5 illustrates a specific but not limiting embodiment of a multi-analyte monitor such as that shown in FIG. 4, with the two sensing schemes located on different planes of the flexible sensor.

FIG. 6 illustrates a specific but not limiting embodiment of a multi-analyte monitor, such as that shown in FIG. 3, with the two sensing schemes located on different planes.

FIGS. 7A-7B show the working principles of the PALS, a non-limiting embodiment of the MAMS.

FIG. 8 illustrates a non-limiting embodiment of the pH sensor sheet (HSS) fabrication for the MAMS.

FIG. 9 is a non-limiting embodiment of the Lactate Oxidase (LOX) sheet fabrication for the MAMS.

FIG. 10 illustrates a non-limiting embodiment of the connections and essential circuit boards of the MAM, the PALS.

FIG. 11 illustrates one non-limiting embodiment of the filter-coated photodetector.

FIG. 12 illustrates one non-limiting embodiment of the flexible sensor of the MAM.

FIG. 13 illustrates a benchtop optical system used to validate the dual excitation, single band detection scheme to monitor pH on the PALS, a non-limiting embodiment of the MAMS.

FIG. 14 is a graph that shows HSS emission spectra after 400 and 465 nm LED excitation as well as the transmission spectra of the Primary Green Filter.

FIG. 15 is a graph that shows the filtered HSS spectra that are detected by the MAM.

FIG. 16 shows a graph modeling the dual excitation, single band detection scheme for pH sensing on the MAMS.

FIG. 17 is a graph of the stability of an embodiment of the pH sensing modality on the multi-analyte flexible sensor.

FIG. 18 is a graph of the rise time of an embodiment of the pH sensing modality on the multi-analyte flexible sensor after a solution exchange from pH 7.45 to pH 7.01.

FIG. 19 is a graph of steady state measurements in a series of pH solutions from an embodiment of the pH sensor on the multi-analyte flexible sensor.

FIG. 20 is a graph of a calibration curve from pH 6.92 to 7.59 from an embodiment of the pH sensor on the multi-analyte flexible sensor.

FIG. 21 is a graph of the stability of an embodiment of the lactate sensing modality on the multi-analyte flexible sensor.

FIG. 22 is a graph of the rise time of an embodiment of the lactate sensor on the multi-analyte flexible sensor after a solution exchange from 0 mM to 6 mM lactate.

FIG. 23 is a graph of an embodiment of the lactate sensor on the multi-analyte flexible sensor measuring a series of lactate solutions to show reversibility.

FIG. 24 is a graph of a calibration curve from 0 mM to 14 mM lactate measured by an embodiment of the lactate sensor on the multi-analyte flexible sensor.

FIG. 25 is a graph of an embodiment of the multi-analyte flexible sensor detecting lactate and pH levels that model hyperlactatemia.

FIG. 26 is a graph of an embodiment of the multi-analyte flexible sensor detecting lactate and pH levels that model lactic acidosis.

FIG. 27 is a graph of an embodiment of the multi-analyte flexible sensor detecting lactate and pH levels that model extreme metabolic alkalosis.

FIG. 28 is a graph of an embodiment of the multi-analyte flexible sensor detecting lactate and pH levels that model respiratory alkalosis and hyperlactatemia.

FIG. 29 is a graph of an embodiment of the multi-analyte flexible sensor detecting lactate and pH levels that model respiratory acidosis.

FIG. 30 is a photograph of the PALS embodiment of the MAMS implanted in a rabbit to measure pH and lactate in a model of hypoxemia.

FIG. 31 is a graph of an embodiment of the multi-analyte flexible sensor tracking pH in vivo in a rabbit model of chlorine gas poisoning and cobinamide treatment, with reference measurements from an ABBOTT i-STAT.

FIG. 32 is a graph of an embodiment of the multi-analyte flexible sensor tracking lactate levels in vivo in a rabbit model of chlorine gas poisoning and cobinamide treatment, with reference measurements from an ABBOTT i-STAT.

FIGS. 33A-33B show two flex circuits placed side-by-side. In FIG. 33A, the top flex circuit has an LED that emits light. The bottom flex circuit has an LED that acts as a photodiode or photodetector. FIG. 33B shows a polytetrafluoroethylene (PTFE) sheet placed over the emission-LED/detection-LED pair.

FIG. 33C shows the detection-LED wired into an existing transimpedance operational-amplifier circuitry, where this circuit was originally designed for a photodiode.

FIG. 33D shows a time-matched graph showing the emission-LED input (or drive) signal (labeled “LED Input Signal”) and detection-LED (labeled “Photodiode Signal”) response measured by an oscilloscope.

### DETAILED DESCRIPTION OF THE INVENTION

Following is a list of elements corresponding to a particular element referred to herein:
optoelectronic component 101
flexible circuit 102
recording unit 103
tissue 104
light source 301
photodetector 105, 302, 303
wires or traces 201
conductive pad 202, 203
sheet 304, 305

### List of Abbreviations

MAM—multi-analyte monitor
YSI—Yellow Spring Instrument
PALS—pH and Lactate Sensor
HPTS—8-Hydroxypyrene-1,3,6-trisulfonic acid trisodium salt
HSS—pH Sensor Sheet
PEGDMA8000—poly(ethylene glycol) dimethacrylate 8000
PTFE—polytetrafluoroethylene
LOX—Lactate Oxidase
LED—Light emitting diode
1× PBS—1× Phosphate Buffered Saline Solution
ANOVA—analysis of variance
CV—coefficient of variance

Herein “analytes” and “biomarkers” are used interchangeably. Examples include the non-limiting list of molecules, ions, hormones, carbohydrates, proteins, organic acids and bases, nucleic acids, lipids and the like. pH is also considered an analyte for the purposes of this application because it is related to the concentration of hydrogen ions. Additional measurable biomarkers include concentrations, pressures (such as oxygen and carbon dioxide partial pressures), temperature, electric potential, current, resistance, capacitance, and the like. Thus, analyte/biomarkers are terms to denote any substance that can be monitored.

The word “luminescent” may be used as an adjective to describe a substance or molecule that upon excitation with an energy source (e.g. light), operates by changes in absorption, scattering, polarization, refractive index, reflection, diffraction, fluorescence, phosphorescence, chemiluminescence, bioluminescence, and the like. This is a non-limiting list of properties.

Herein, when referring to luminescent probe, luminescent dye, protein, and molecule, these terms describe a luminescent molecule, and may thus be used interchangeably. In some cases, protein and molecule may refer to non-luminescent molecules and/or an analyte.

Herein, optode refers to a non-limiting type of sensing modality that utilizes and/or measures light or uses a measurement of a property of light as part of a scheme for sensing an analyte.

Herein, when referring to light sources, these components emit photons.

Herein, when referring to photodetectors (105, 302, 303), this includes the non-limiting list of photodiodes, phototransistors, photoresistors, photomultiplier tubes, avalanche photodiodes, Schottky photodiode, pyroelectric detectors, and the like. These components detect photons and convert them to a measurable read out, including, but not limited to current and resistance. Opto-electronic components include light sources and detectors. Detectors and photodetectors may be used interchangeably.

Herein, the term “sheet” is a general term that can refer to a film, a matrix, a coating, a covering, a layer, a topping, a surface, a substance, or a substrate; and may be used interchangeably therewith. As such, a “sheet” is not limited to just a flat form, but can include other forms. As used herein, the sheet, such as the sensor sheet (304, 305), includes anything that is placed on or proximal to at least one opto-electronic component that is used to provide signals sensitive to an analyte. For example, a sensor sheet (304, 305) may include anything that comprises a luminescent dye.

Herein, the terms “catalyst” and “enzyme” are used interchangeably.

According to some embodiments, the present invention features a pH sensor sheet (305) comprising a sheet coated with a polymer coating comprising particles and pH sensitive dye molecules bound thereto. In one embodiment, the sheet has pores that allow for the particles to penetrate into the sheet. In another embodiment, the sheet has pores that are smaller than the particles, thereby preventing the particles from penetrating into the sheet. In one embodiment, the sheet may be comprised of hydrophilic polytetrafluoroethylene. In some embodiments, the pH sensitive dye molecules are ionically bound to the particles. In some embodiments, the particles are resin beads. In some embodiments, the polymer coating further comprises a hydrogel.

According to other embodiments, the present invention features a pH sensor comprising a support substrate, a plurality of opto-electronic components comprising at least one light source (301) disposed on a surface of the support substrate, and at least one detector (302) disposed on the surface of the support substrate, adjacent to the at least one light source (301), and a pH sensor sheet (305) disposed on the opto-electronic components. In some embodiments, the pH sensor sheet (305) may comprise a sheet having a surface coated with a polymer coating comprising particles and pH sensitive dye molecules bound thereto. The at least one light source (301) may be configured to illuminate the pH sensitive dye molecules, which produces a luminescence that is detectable by the at least one detector (302).

In some embodiments, the opto-electronic components may further comprise a second light source (301) disposed on the surface of the support substrate. The pH sensor sheet (305) can be further disposed on the second light source (301). The second light source (301) is configured to illuminate the pH sensitive dye molecules. In one embodiment, the second light source (301) may be positioned such that the at least one detector (302) is disposed between the two light sources (301). The at least one detector is coated with an optical filter to pass a specific band or bands of wavelength.

In other embodiments, the opto-electronic components may further comprise a second detector (302) disposed on the surface of the support substrate. The second detector (302) is configured to detect the luminescence of the pH sensitive dye molecules. The pH sensor sheet (305) may be further disposed on the second detector (302). The second detector (302) may be positioned such that the at least one light source (301) is disposed between the two detectors (302). In some embodiments, each detector (302) may be coated with an optical filter to pass a specific band or bands of wavelength. The optical filter of one detector may be the same or different from the optical filter of the second detector.

In one embodiment, the pH sensor sheet (305) comprises a single sheet disposed on the plurality of opto-electronic components. In another embodiment, the pH sensor sheet (305) comprises a plurality of sheets. Each sheet may be disposed on one of the opto-electronic components. In some embodiments, the plurality of sheets is coated with the same polymer coating. In other embodiments, the plurality of sheets is coated with different polymer coatings.

In some embodiments, the coated surface of the pH sensor sheet is facing the opto-electronic components. In one embodiment, the sheet has pores that allow for the particles to penetrate into the sheet. In another embodiment, the sheet has pores that are smaller than the particles, thereby preventing the particles from penetrating into the sheet.

In some embodiments, the support substrate of the pH sensor may comprise a flexible base layer. The support substrate may be a thin, flat layer comprised of a flexible material. Electrically conducting wires or traces (201) may be disposed on the flexible base layer and operatively coupled to the opto-electronic components. In other embodiments, the support substrate is a flexible circuit operatively coupled to the opto-electronic components. In some embodiments, the plurality of opto-electronic components share electrically conducting wires or traces (201), thereby reducing a total number of electrically conducting wires or traces (201) required. The shared electrically conducting wires or traces (201) enable the plurality of opto-electronic components to be operated one at a time or simultaneously. For example, the plurality of opto-electronic components further comprise a plurality of light sources that can be activated one at a time to limit any optical signal crosstalk. In other embodiments, the shared electrically conducting wires or traces (201) enable the plurality of opto-electronic components to operate or receive current from a current source.

In some embodiments, the pH sensor may further comprise a recording unit (103) operatively coupled to the opto-electronic components. The recording unit (103) contains internal circuitry for activating or supplying power to the opto-electronic components. The recording unit (103) may be configured to receive and record luminescence signals from the at least one detector (302). The recording unit (103) includes a printed circuit that is operatively coupled to the opto-electronic components.

In some embodiments, the pH sensor is configured to be implanted under skin, in a tissue, or in an organ. In other embodiments, the recording unit (103) is configured to be placed on the skin or implanted under the skin, in the tissue, or in the organ.

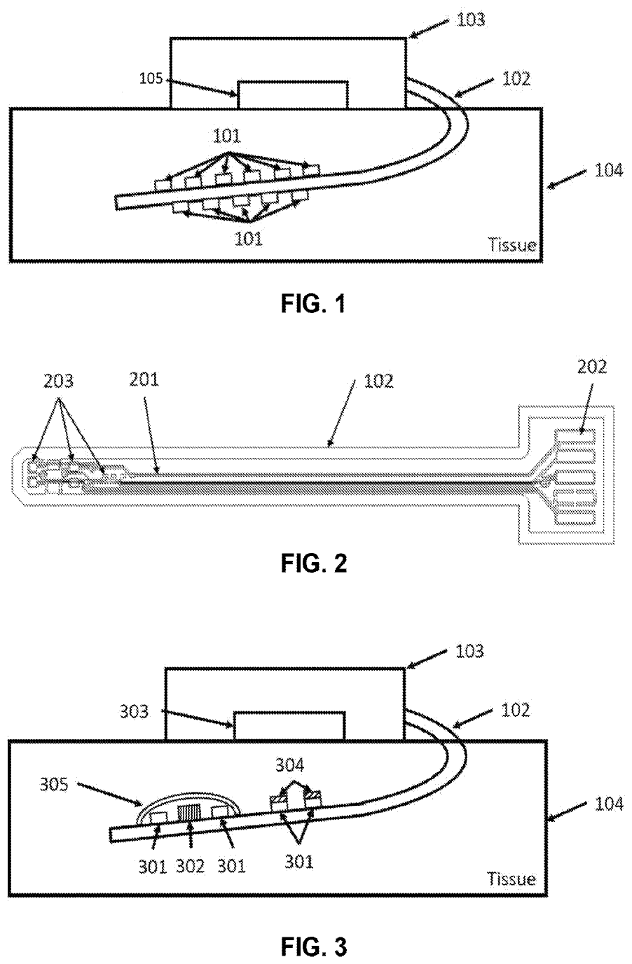

In some embodiments, the present invention features a multi-analyte monitoring (MAM) system comprising a multi-analyte sensor unit and a recording unit (103) operatively coupled to the sensor unit, as shown in FIGS. 1-6. In some embodiments, the multi-analyte sensor unit may comprise a support substrate, a plurality of analyte sensors disposed on the support substrate, the plurality of analyte sensors configured to monitor a plurality of analytes, each analyte sensor comprising at least one opto-electronic component disposed on the support substrate, and a luminescent sheet disposed on the at least one opto-electronic component. The luminescent sheet may comprise a porous sheet coated with a dye coating. The recording unit (103) can include an analyte detector (303) for detecting luminescence from at least one of the analyte sensors. In some embodiments, the at least one opto-electronic component is a light source (301). In some embodiments, the plurality of analyte sensors may include the pH sensor as described herein.

In some embodiments, the luminescent sheet may comprise one or more catalysts configured to interact with a target during interaction with an analyte, and a luminescent dye configured to interact with the target and generate a luminescence corresponding to said analyte. The changes in said luminescence may be related to the concentration of said analyte. In some embodiments, the one or more catalysts may consume the target in the presence of the analyte and the luminescent dye. The target binds or reacts to the luminescent dye and at least partially quenches the luminescence of the luminescent dye, thereby resulting in the change in luminescence. In one embodiment, the target is oxygen. In another embodiment, the one or more analytes are lactate, glucose, ketones, or a combination thereof.

In other embodiments, the one or more analyte sensors may further comprise luminescent probes. Each luminescent probe may comprise a fluorescent protein pair. Each fluorescent protein is conjugated to a macromolecule. A binding event produces an energy transfer between the fluorescent protein pairs that yield luminescent signals. In some embodiments, the luminescent probes are Förster Resonance Energy Transfer (FRET) probes.

In some embodiments, the luminescent sheet comprises a single sheet disposed on the at least one opto-electronic component. In one embodiment, the luminescent sheets of at least two of the analyte sensors may comprise the same dye coating. In another embodiment, the luminescent sheets of the analyte sensors comprise different dye coatings.

In some embodiments, the coated surface of the porous sheet is facing the opto-electronic components. In one embodiment, the sheet has pores that allow for the particles to penetrate into the sheet. In another embodiment, the sheet has pores that are smaller than the particles, thereby preventing the particles from penetrating into the sheet.

In some embodiments, the support substrate comprises a flexible base layer. The support substrate may be a thin, flat layer comprised of a flexible material. The support substrate may include electrically conducting wires or traces (201) disposed on the flexible base layer and operatively coupling the recording unit (103) to the analyte sensors. In other embodiments, the support substrate is a flexible circuit operatively coupling the recording unit (103) to the analyte sensors.

In some embodiments, the opto-electronic components of the plurality of analyte sensors share electrically conducting wires or traces (201), thereby reducing a total number of electrically conducting wires or traces (201) required. In one embodiment, the shared electrically conducting wires or traces (201) enable the opto-electronic components to be operated one at a time or simultaneously. In another embodiment, the shared electrically conducting wires or traces (201) enable the opto-electronic components to operate or receive current from a current source. For example, the opto-electronic components comprise light sources that can be activated one at a time to limit any optical signal crosstalk. In some embodiments, the recording unit (103) contains internal circuitry for activating or supplying power to the sensor unit. The recording unit (103) may be configured to receive and record signals that correspond to the luminescence detected by the analyte detector (303).

In one embodiment, the analyte sensors are disposed on one side of the supporting substrate. In another embodiment, the analyte sensors are disposed on both sides of the supporting substrate.

In some embodiments, the sensor unit is configured to be implanted under skin, in a tissue, or in an organ. In further embodiments, the recording unit (103) is configured to be placed on the skin or implanted under the skin, in the tissue, or in the organ such that the analyte detector (303) is positioned above or directly facing at least one of the analyte sensors of the sensor unit.

According to other embodiments, the present invention features a method of detecting and monitoring levels of multiple analytes in a subject. The method may comprise providing an embodiment of the MAM system described herein, and implanting at least the sensor unit of the MAM system into the subject. In further embodiments, the method comprises implanting the recording unit (103) into the subject such that the analyte detector (303) is positioned above or directly facing at least one of the analyte sensors of the sensor unit.

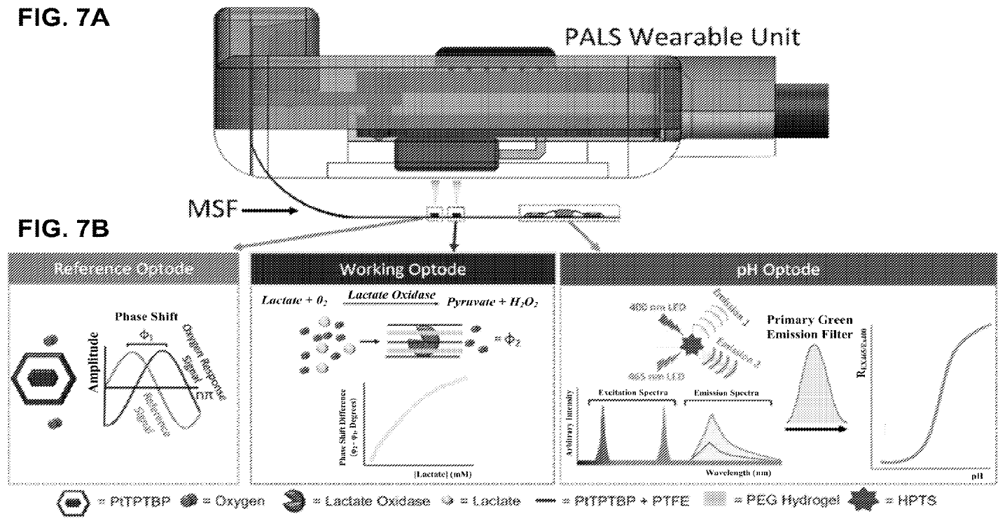

In some embodiments, the MAM sensor may be used to measure both pH and lactate, referred to as the pH and Lactate Sensor (PALS), as shown in FIGS. 3 and 7. PALS employs two unique sensing modalities at the tip of the flexible sensor (102) to monitor pH and lactate: (1) a dual excitation, single band detection scheme that collects pH-sensitive light emissions and (2) a luminescence lifetime detection scheme that captures oxygen and lactate-sensitive light emissions. For the embodiment of PALS, the recording unit (103) is referred to as the wearable unit, and additionally, the wearable unit is connected to a backend controller unit. In an alternate, and in some cases improved embodiment that is non-limiting, the recording unit (103) would contain the electronics found in both the PALS's wearable unit and the backend controller, and this unit can, but need not be, wireless. Such a strategy could be used with all embodiments of this invention. In vitro results, described below, show rapid rise times for both pH and lactate sensing, as well as measurement reversibility, and sensitivity across their respective pathophysiological ranges. Further, when measuring both analytes simultaneously, measurements were shown to be free of crosstalk. An implant study in a rabbit model of hypoxemia provides further evidence that PALS signals are reversible and trend appropriately with reference to a handheld blood gas analyzer.

Below are additional non-limiting embodiments of the present invention, including modifications such as arrangement and composition of opto-electronic components (101), sensing chemistries, and materials as seen in FIGS. 3-6.

### Luminescent Molecules

In the embodiment of the invention described above, an implantable sensor measures both lactate and pH using luminescent molecules. The lactate sensing component comprises a catalyst that consumes oxygen in the presence of an analyte and a luminescent molecule wherein oxygen can bind, react, or bind and react, to the luminescent molecule and at least partially quench the luminescence of the molecule. Quenching results in a measurable change in luminescence measured by the non-limiting examples of changes in luminescence intensity, luminescence lifetime, and luminescence phase change relative to an oscillating excitation light source. The catalyst can be of the non-limiting class of proteins, enzymes, single-stranded nucleic acids, and other macromolecules. The pH sensing component comprises a luminescent molecule that exhibits a change in emission spectrum with changing pH. The pH-sensitive change in the luminescent molecule's emission spectrum may be due to a change in the luminescent dye's absorption spectrum that changes with pH. This change in absorption spectrum may be probed with at least one excitation wavelength. Changes in luminescence can also be due to phenomena including those from the non-limiting list of changes to the luminescent molecule's absorption efficiency, scattering efficiency or polarization. Changes in the luminescent molecule's emission spectrum can be analyzed by examination of the spectrum or using techniques such as, but not limited to monitoring changes in emission intensity across at least one wavelength band.

In some embodiments, an analyte or quantity would be measured by a luminescent probe, whereby the probe is excited at two or more wavelength bands, and emission would be detected at one or more wavelength bands. In one embodiment, at least two LEDs, each emitting the same or different light spectra, would illuminate the luminescent probe sequentially. At least one detector, designed to collect light from at least one spectral band, would return at least two signals, one signal per LED illumination. The ratio of collected spectra can be utilized to monitor an analyte. Such a strategy was implemented for pH sensing with PALS. However, in general these luminescent probes, in the presence of an analyte, can exhibit a change in emission spectra, temporal dynamics, and/or total intensity when excited by either multiple wavelength bands or a particular light spectrum, including a narrow spectrum such as delivered by a laser. Such detectable phenomena can result from analyte-specific changes to luminescent probe optical absorption at various wavelengths or analyte-specific changes to luminescent probe light emission. In some cases, the luminescent probe serves as a scattering or absorbing substance, where transmitted or reflected (back-scattered) light from a light source (301) is the measured signal. In all these embodiments, the photodetector's photocurrents or photovoltages can be used to compute a calibrated function to the analyte.

For the case of sensing quantities such as pH or for specific elements, the luminescent probe can be, but is not limited to, 8-Hydroxypyrene-1,3,6-trisulfonic acid trisodium salt (HPTS), N-[2-[(acetyloxy)methoxy]-2-oxoethyl]-N-[5-[2-[2-[bis[2-[(acetyloxy)methoxy]-2-oxoethyl]amino]-5-methylphenoxy]ethoxy]-2-[(5-oxo-2-thioxo-4-imidazolidinylidene)methyl]-6-benzofuranyl]-(acetyloxy)methyl ester Fura (Red™, AM, cell permeant), Furaptra (Mag-Fura-2) Tetrapotassium Salt, Sodium-binding Benzofuran Isophthalate Acetoxymethyl ester (SBFI, AM), mitochondria-targeted fluorescent sensor (Mito-ST), 4-[2-[6-(Dioctylamino)-2-naphthalenyl]ethenyl]-1-(3-sulfopropyl)-pyridinium, inner salt (di-8-ANEPPS), 2′,7′-bis-(Carboxyethyl)-5-(and-6)-carboxyfluorescein (BCECF), green fluorescent protein-based sensors (pHluorins), 5,5,6,6′-tetrachloro-1,1′,3,3′ tetraethylbenzimi-dazoylcarbocyanine iodide (JC-1), Sypher, pHrodo Green, Phrodo Red, Fluoresceins and carboxyfluoresceins, Oregon Green, or Bis-fura-2t.

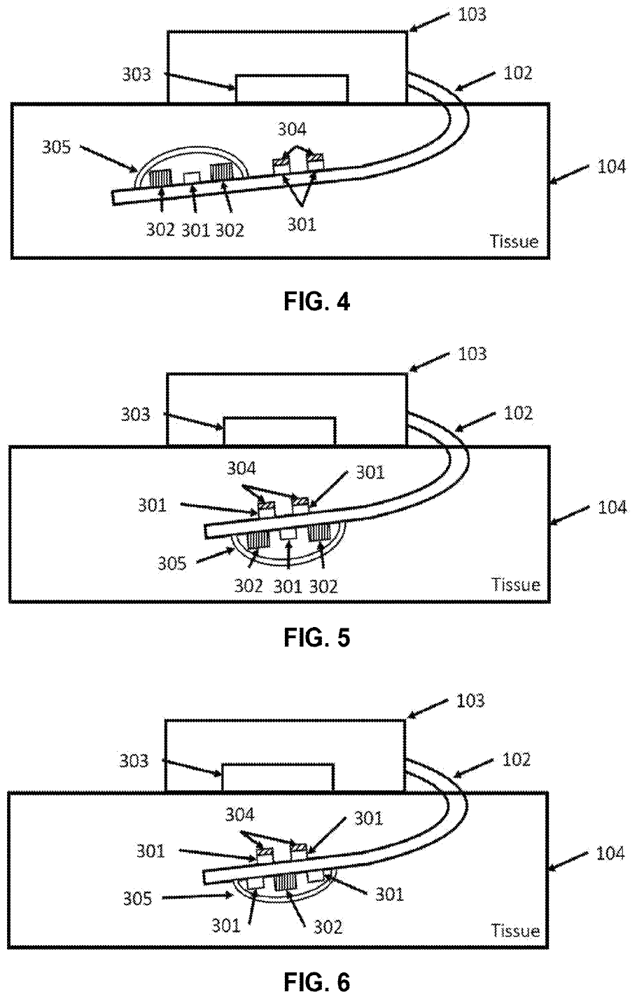

In some embodiments, an analyte or quantity would be measured by a luminescent probe, whereby the probe is excited by at least one wavelength band, and emission would be detected, such that the emission spectra is segregated into at least two wavelength bands. In one simple embodiment a single LED emitting light with a specific light spectrum would illuminate a luminescent probe. At least two detectors, each designed to collect light from at least one spectral band, would return at least two signals, one per each detector. These embodiments could be, but are not limited to, as illustrated in FIGS. 4 and 5. These luminescent probes, in the presence of an analyte, can exhibit a change in emission spectra, temporal dynamics, and/or total intensity when excited by either multiple wavelength bands or a particular light spectrum, including a narrow spectrum such as delivered by a laser.

A non-limiting list of such probes includes 2,3-Dicyanohydroquinone (DCH) for sensing pH, LysoSensor™ Yellow/Blue DND-160 for sensing pH, 5-(and-6)-Carboxy SNARF (SNARF) for sensing pH, and Indo-1 for sensing calcium. In a non-limiting embodiment, these probes would be excited by a single LED, and light would be collected by two photodetectors (302), each coated with at least one optical filter to pass a specific band or bands of wavelength. The photodetectors' photocurrent can be used to compute a calibrated function to the analyte. In other embodiments, each of the photodetectors (302) can have innate properties of filtering specific band(s) of wavelengths, or a single detector can be utilized that can detect at least one or more spectral bands. A non-limiting example of such a detector is the buried double junction photodetector.

In some embodiments, at least two luminescent probes are conjugated to one or multiple macromolecules such as ones from the class of proteins, or specifically, but non-limiting, such as an analyte-binding protein or analyte-sensitive protein, specific to an analyte including, but not limited to peptides, sugars, lipids, hormones, nucleic acids, calcium, magnesium, zinc, and other ions or electrolytes. Such macromolecules may also be, but not limited to, of the class of nucleic acids, nucleic acid aptamers, antibodies, complex carbohydrates, nanobodies, lipids, and non-naturally occurring molecular species. In such embodiments, the result of a binding event is an energy transfer between at least two of the luminescent probes that yield calibratable signals. This non-limiting strategy could also make use of a non-luminescent quencher. One specific category of such macromolecules are Förster Resonance Energy Transfer probes, naturally occurring or engineered to produce a FRET signal specific to an analyte. Examples of FRET probes include but are not limited to, the proteins Twitch2B, Chameleon, Zapcy, Laconic, MARIO, CYPHR, DAGR, ficaro, CitA, FLIPE, TSMOD. Such FRET probes would comprise luminescent probes and or quenchers that can be of the class of genetically engineered fluorescent proteins including but not limited to the green fluorescent protein, cyan fluorescent protein, yellow fluorescent protein, red fluorescent protein, or others from a growing catalog of fluorophores. The fluorescent proteins and FRET construct can also be novel engineered constructs.

Additionally, in some embodiments, the fluorophores and or quenchers can be non-protein molecules including but not limited to Alex Fluor, Atto, Brilliant, Cellbrite, CF, Chromeo, Coralite, Dylight, Efluor, HiLyte, iFluor, IRDye, iRFP, Janella Fluor, Live-or-Dye, LysoView, MaxLight, MemBrite, MitoView, Neurovue, NL, NovaBlue, NovaRed, NovaYellow, Nuclear-ID, Oyster, PromoFluor, Qdot, ReadiLink, Seta dyes, Spark, STAR, SureLight, ViaKrome, VivaFix dyes. Similar embodiments to FRET that can be considered for the sensor include, but are not limited to, bioluminescence resonance energy transfer, phosphorescence resonance energy transfer, and combinations of these modalities.

In some embodiments, a first analyte-sensitive luminescent molecule emits light differentially with analyte concentration, and a second luminescent molecule is insensitive to that analyte. In this case, the second luminescent molecule serves as reference to the first luminescent molecule.

In some embodiments, an oxygen sensitive luminescent molecule can be utilized in conjunction with an oxidase to measure analytes including, but not limited to lactate, glucose, and ketones. The luminescent molecules can be used to measure oxygen, and the like. The luminescence molecule can be phosphorescent; however, this is not a limiting case. Other cases would include molecules that are of the class, but not limited to the class of molecules that are fluorescent, bioluminescent, and chemiluminescent.

In the case of an oxidase-based sensing strategy, an oxygen-sensitive phosphorescent molecule can be used. Such molecules include, but are not limited to, platinum(II) meso-tetraphenyltetrabenzoporphoryn (PtTPTBP) and other metallo-porphyrins such as platinum octaethylporphyrin (PtOEP), platinum octaethylporphyrin (PdOEP), platinum(II) meso-tetra (pentafluorophenyl)porphine (PtTFPP), palladium(II) meso-tetra(pentafluorophenyl)porphine (PdTFPP), platinum(II) octaethylporphyrinketone (PtOEPK), palladium(II) octaethylporphyrinketone (PdOEPK), palladium(II) meso-tetra phenyltetrabenzoporphyrin (PdTPTBP), platinum(II) meso-tetra(4-fluorophenyl)tetra benzoporphyrin (PtTPTBPF), palladium(II) meso-tetra(4-fluorophenyl)tetrabenzo porphyrin (PdTPTBPF), platinum(II) meso-tetra(4-fluorophenyl)mononaphthotribenzo porphyrin (Pt1NF), palladium(II) meso-tetra(4-fluorophenyl)mononaphthotribenzo porphyrin (Pd1NF), platinum(II) meso-tetra (4-fluorophenyl)mononaphthotribenzo porphyrin (cis-Pt2NF), cis-palladium(II)-meso-tetra(4-fluorophenyl) dibenzodinaphtho porphyrin (Pd2NF), platinum(II) meso-tetra(4-fluorophenyl) monobenzotrinaphtho porphyrin (Pt3NF), palladium(II) meso-tetra(4-fluorophenyl)monobenzotrinaphtho porphyrin (Pd3NF), platinum(II) tetraphenyltetranapthoporphyrin (PtTPTNP), palladium(II) meso-tetraphenyltetranaphtho porphyrin (PdTPTNP), platinum(II) tetrabenzo porphyrin-octabutylester (PtTBP(CO2Bu)8), palladium(II) tetrabenzoporphyrin-octabutyl ester (PdTBP(CO2Bu)8), platinum(II) aza-triphenyltetrabenzoporphyrin (PINTBP), platinum(II) aza-triphenyltetrabenzoporphyrin (PdNTBP).

For the case of a sensor that is implanted under the skin or any tissue compartment, these luminescent molecules can emit light in the visible spectrum, but it may be beneficial to select luminescent molecules that emit in the near infrared spectrum because such wavelengths exhibit relatively less scatter and absorption in tissue, a property that is essential if light is detected when tissue is between the detector and luminescent molecule. In some embodiments, the flexible sensor (102) is placed within an organ and the recording unit (103) is placed on the surface of the organ, just above the sensor. Also, both the sensor and recording unit (103) can be placed within the same tissue.

### Light Sources

Also disclosed herein are examples of light sources for the multi-analyte sensor. In general, a light source can be any object or source that emits photons. Such light sources include, but are not limited to, light emitting diodes (LEDs), lasers, vertical cavity surface emitting lasers, quantum dots, lamps, luminescent molecules, electroluminescent wires, coherent light sources, and incoherent light sources.

### Photodetectors

Also disclosed herein are examples of photodetectors (105, 302, 303) for the multi-analyte sensor. In this non-limiting embodiment, photodetectors (105, 302, 303) are components that collect photons and convert them into measurable parameters such as, but not limited to, current, voltage, and resistance. Such a detector must be sensitive to the range of wavelengths specific to the analyte-sensitive luminescent molecules, or in general to light that is emitted as part of the sensing scheme. Typically, such detectors operate by converting photons to either a current or voltage that can be sampled or recorded using data acquisition strategies such as analog to digital converters. Such detectors include but are not limited to photovoltaic and photoconductive detectors. As well, the detectors may be in the non-limiting forms of a throughhole, surface mount, and open access embodiments. The working principles of the photodetector include, but are not limited to, NPN, PNP, PN and PIN photodetection (105, 302, 303). In some embodiments, the photodetector is a photomultiplier tube, avalanche photodiode, Schottky photodiode, and pyroelectric detector. In some embodiments, the detector is lined with a pinhole to limit the angle of acceptance of light as received by the detector.

In one embodiment of the invention, the photodetector (105, 302, 303) is a silicon photodiode. In a further embodiment, the silicon photodiode (105, 303) is placed within the recording unit (103) either worn on top of the skin, or in a scheme by which the luminescent molecule(s) and silicon photodiode (302) are separated by tissue. In another embodiment of the invention, this silicon photodiode(s) (302) is placed onto the implantable flexible sensor (102). In this case, the silicon photodiode (302) must be of comparable size to the flexible sensor (102) width, which is on the order of 50 μm to 500 μm, but these sizes are not limiting. In a further embodiment, multiple silicon photodiodes (302) are located in some combination on the flexible sensor (102) and on/in the recording unit (103). Further, these examples are not limited to silicon photodiodes, but applicable to any photodetector.

### Detection Strategies

Disclosed herein are embodiments of a sensor system that comprises a catalyst that consumes oxygen in the presence of an analyte and a luminescent molecule wherein oxygen can bind, react, or bind and react, to the luminescent molecule and at least partially quench the luminescence of the molecule. Quenching results in a measurable change in luminescence. Such light emission can also exhibit a change in emission spectra, temporal dynamics, and/or total intensity. Temporal dynamics can be measured using metrics such as luminescence lifetime, and luminescence phase change relative to an oscillating standard, such as the excitation light source. The luminescent molecule and protein are combined within a sensing matrix (304) that is adhered to a light source. Another sensing matrix (304), with or without an oxygen-consuming protein, is placed upon a separate light source that emits light at the same wavelength to be used as a reference.

In one embodiment, the sensor can contain a luminescent probe that exhibits an emission spectra and peak intensity that may be specific to the wavelengths being emitted by at least two different light excitations, which can be correlated to the concentration of an analyte. The change in the luminescence output may be due to, but not limited to, a change in absorption efficiency at multiple wavelengths due to a change in analyte concentration. The ratio of emission spectra or intensity after at least two light source excitations can be correlated to the concentration of the analyte. Taking ratios of these emission spectra is only one example of analysis, alternative mathematical treatments may be appropriate. In some embodiments, as described above, the luminescent probe is immobilized within a sensing matrix (304, 305) that is then adhered to the sensor, covering the necessary light source and detector components as shown in FIGS. 3-6.

In one embodiment, the flexible sensor (102) can be configured for luminescence energy transfer-based sensing. In this case, the flexible sensor (102) can contain at least one light source, at least two detectors, and a luminescent probe, contained within a matrix (304, 305), that emits a light signal with at least two peak-wavelengths when excited by at least a single wavelength spectrum. The matrix (304, 305) can cover, be adhered to, or overlap, all the light sources and detectors. The matrix (304, 305) may also cover, be adhered to, or overlap a subset of light sources and detectors. In some embodiments, the ratio of the peak luminescence emission intensities can be modulated by an analyte's concentration, and when excited by the light source can be collected with the photodetectors (302) to yield a calibratable signal. Additionally, properties of emitted light such as the non-limiting examples of area under the curve, isosbestic points, and spectra slopes, can be utilized to quantify the analyte concentration. In all cases, calculating ratios is only one example of analysis, alternative mathematical treatments may be appropriate. As mentioned above, the light sources and detectors may be on the same plane on the flexible sensor (102) or on opposite sides of the flexible sensor (102). As well, these opto-electronic components (101) can be placed adjacent to one another along the short axis of the flexible sensor or in a linear arrangement along the long axis of the flexible sensor (102). Additional embodiments of the layouts have been described above.

In one embodiment, the sensing technology does not rely only on at least one analyte-specific exogenous luminescent sensing molecule, but instead relies on at least one endogenous molecule. Here, an optode can be defined as comprising at least two light sources (301) or two spectral-bands of excitation light and at least one detector (302) that measure light across at least one spectral band, in order to monitor an analyte. This embodiment relies on optical properties of an analyte or products of a chemical reaction that produces or consumes that analyte. In other embodiments, the analyte itself contains the optical properties. Such properties could include, but are not limited to quantum efficiency of absorption, absorption spectra and associated quantum efficiencies, light scatter, polarization modification, and changes in the phase of an oscillating light source. In other embodiments a complementary reference optode is used along with that described above. The reference optode may comprise, in a non-limiting embodiment, a set of light source(s) (301) and detector(s) (302) that probe a volume absent of the molecule(s) that provides signal in the first optode. As a non-limiting example, the working optode comprises a light source (301) that would illuminate a matrix (304) containing a sensing molecule that consumes the analyte and produces at least one product that has optical properties distinct from the analyte itself. In this case, this reference optode could be identical to the working optode with the exclusion of the sensing molecule. In other embodiments no exogenous molecules are included in the optodes, and the optical properties of only endogenous molecules are used. One limiting example would include optodes for measuring hemoglobin oxygen saturation, in which light sources from at least two distinct wavelength bands could be used in conjunction with at least one light detector.

In some embodiments of the MAM, the sensing strategies (mentioned above) can be deployed as a stand-alone wireless unit that can be implanted in a tissue. In these embodiments, both the recording unit and the flexible sensor can be implanted in tissue. The sensor may be powered wirelessly through the tissue, with an internal battery, or may be powered with a connected power source. In this embodiment, data may be transferred wirelessly to a device outside the tissue.

### Optical Filter Application

Also disclosed herein are embodiments of filter applications onto an opto-electronic component (101). In some non-limiting embodiments, the filter can be made of an absorptive dye, or an absorptive dye immobilized within glass, polyester, resin, acrylic, or gelatinous substrate. In some non-limiting embodiments, the filter is a dielectric filter, long pass filter, bandpass filter, multi bandpass filter, dichroic filter, dichroic mirror, polarizer, polarization filter, notch filter, color filter, neutral density filter, or an absorptive filter. In some embodiments, the filter is applied using refractive index-matching glue and similar adhesives. In some embodiments, the filter is applied onto the opto-electronic component (101) via a thermal process, including but not limited to melting, baking, dip coating, and hot embossing. In some embodiments, a filter is applied onto the opto-electronic component (101) via methods including, but not limited to, a deposition process such as vacuum thermal evaporation, electron beam evaporation, laser beam evaporation, arc evaporation, molecular beam epitaxy, ion plating evaporation, DC sputtering, RF sputtering, chemical bath deposition, spray pyrolysis technique, plating, electroplating technique, electroless deposition, chemical vapor deposition, low pressure vacuum deposition, plasma enhanced vacuum deposition, and atomic layer deposition. In some embodiments, more than one optical filter or a mixture of optical filters is adhered onto an opto-electronic component (101).

### Flexible Sensors

Also disclosed herein are material embodiments for the flexible sensor (102). In some embodiments, the flexible sensor (102) substrate comprises, but is not limited to, Flame retardant (FR)-1 through FR-6, Composite epoxy materials (CEM)-1 through CEM-5, Garolite (G)-10, G-11, aluminum, polyethylene terephthalate, polytetrafluoroethylene, polyimide, pyralux, and kapton. In some embodiments, the flexible sensor (102) is made using techniques common to flexible circuits (102), and can be a flexible circuit (102). Such techniques include layer-by-layer fabrication and incorporate vias allowing multiple conductive layers separated by insulating layers, where conductive layers can be connected to one another through the vias. Non-limiting examples of the trace material include aluminum, metal, iron, and copper, and while non-limiting examples of the insulating layers include made of urethane, acrylic, epoxy, and silicone chemistries. In non-limiting embodiments, the flex circuit layering and patterning is completed in large sheets followed by sensor individuation via methods such as laser cutting, milling, jigsaw, table saw, fine-toothed saw, stamping, and the like. In other embodiments, the flexible circuit (102) need not be fabricated as a flexible circuit (102) but may contain other types of conducting paths made of, for example, wires that are organized in a polymer matrix.

### Protective Coatings

Also disclosed herein are embodiments for coating the sensor to prevent sensor damage from aqueous, thermal, mechanical, electrical, and humidity damage. In some non-limiting embodiments, the protective coating contains chemicals such as cyanoacrylate, silicon, diphenylmethane diisocyanate, isoprene, butyl isoprene, nitrile isoprene, styrene butyl isoprene, butadiene isoprene, polyurethane, acrylic, methacrylic acids, epoxies, polyether polyols, polychloroprene, styrene, polyisobutylene, polysulfide, polyamide, styrene-acrylic copolymer, styrene-acrylic emulsion polymer, styrene butadiene, resin and hardeners, or a combination of these materials. Non-limiting commercial examples of the protective coatings include Loctite EA E-60NC, Loctite E-40FL, Loctite E-60HP, Loctite E120-HP, Gorilla Glue and Loctite Marine Epoxy. The protective coating can cure immediately or after a duration of time. The protective coating can be made of biocompatible materials and can function to reduce the foreign body response as compared to a non-biocompatible material. A function of the protective coating is also electrical insulation of all on-sensor electrical or opto-electrical components (101), and insulation of the junction between these components and the flexible sensor (102). In certain embodiments, sensing components, such as the sensing sheets (304, 305) described herein are adhered to the protective coating and not directly to the opto-electronic components (101).

### Sensor Sheet Materials

Sensor sheets (304, 305) contain materials with functions that include, but are not limited to, immobilizing or entrapping luminescent molecules, macromolecules, or other sensing chemistries. The material can also serve as a molecular-weight-cutoff filter for size selection and/or a diffusional barrier. A non-limiting list of such materials includes polyethylene glycol (PEG), poly(ethylene oxide), poly(3,4 ethylenedioxythiophene), bis-poly(ethyleneglycol) lauryl terminated, polyethylene glycol monodisperse solution, 4arm-PEG, catalase-polyethylene glycol, O,O′-Bis[2-(N Succinimidyl-succinylamino)ethyl] polyethylene glycol, poly(ethylene glycol) methyl ether methacrylate, diethylene glycol butyl ether methacrylate, poly(ethylene glycol) methacrylate, poly (silicone-alt-PEG] dimethacrylate, poly(ethylene glycol) dimethacrylate, tetraethylene glycol dimethacrylate, tetra(ethylene glycol) diacrylate, Tri(ethyleneglycol) diacrylate, poly(ethylene glycol) diacrylate, PEG-polyisobutylene, PEG-poly(c-benzyl L-glutamate), polyacrylamide (PAAm), poly(N-isopropylacrylamide) (PNIPAAM), PEG-PNIPAAM, collagen, fibrin, alginate, agarose, dextran, poly(hydroxyethyl methacrylate) (PHEMA), poly(vinylpyrrolidone) (PVP), pullulan, poly lactic-co-glycolic acid (PLGA), poly(butylene terephthalate), poly(2-ethyl-2-oxazoline) PCL, Poly(l-lactic acid) (PLLA) and poly(d-lactic acid) (PDLA), poly(vinyl alcohol) (PVA), chitosan-PVA, gelatin, albumin, polysaccharides, polyesters, polyamides, poly(oligoethylene glycol methacrylate) (POEGMA), poly(vinylpyrrolidinone), phosphorylcholine, poly(-oxazoline), polyethylenimine (PEI), poly(acrylic acid), polymethacrylate, polyelectrolytes, hyaluronic acid, chitosan-based hydrogels, chondroitin sulfate, methylcellulose, hydroxypropylcellulose, hydroxypropylmethylcellulose, carrageenan, amylopectin, amylose, hydroxyethyl methacrylate (HEMA), 2-HEMA, poly(2-HEMA), poly-L lysine, poly(propylene fumarate) cellulose derivatives, elastin, calcium polyphosphate, or a combination thereof. These materials may also contain functional groups belonging to the non-limiting list including hydroxyl groups, sulfhydryl groups, amine groups, n-hydroxysuccinimide groups, thiol groups, hydrazide groups, carboxylic acid groups, biotin groups, streptavidin groups, and hydrazide groups, being of also 8-arm, homobifunctional, or heterobifunctional groups that crosslink via chemical reactions.

In some embodiments, the sensor sheet (304, 305) can contain at least one non-limiting second component, termed here as the substrate. In some embodiments, the substrate is a hydrophilic or hydrophobic material. In some embodiments, the substrate is a porous material. In some embodiments, the substrate is a continuous material. In some non-limiting embodiments, the substrate comprises polytetrafluoroethylene (PTFE), nylon, polyester, polyvinylidene fluoride, polypropylene, alumina oxide, ceramics, ethylene tetrafluoroethylene, fluorinated ethylene-propylene, perfluoro-alokoxy, polycarbonate, cellulose acetate, polyacrylonitrile, polyether ether ketone, polyethersulfone, and the like or a combination of thereof. In non-limiting embodiments, the multiple sensor sheets (304, 305) (with their substrate and respective chemistries) are combined to create a composite material. In non-limiting embodiments, the composite materials are combined via physical compression of the base materials, adhesions of the sensor sheets with an adhesive, simultaneous material polymerization, or a combination thereof. A list of adhesive materials and reagents has been previously described. For simultaneous material polymerization, a non-limiting example is when the materials contain a crosslinking agent (listed in the following sections) that upon either ultra violet, chemical, or thermal crosslinking, fuse the multiple sensor sheets to yield the composite material.

### Crosslinking Embodiments

In some embodiments, a photoinitiator is used to encapsulate a protein, a luminescent dye, and the like using light-activated-polymerizable materials. In some embodiments, the photoinitiator is either hydrophilic or hydrophobic to varying degrees. In some embodiments, the photoinitiator absorbs light in the visible and/or ultraviolet region of light. In some embodiments the photoinitiator can be, but is not limited to, lithium phenyl-2,4,6-trimethylbenzoylphosphinate, 4′-hydroxyacetophenone, 3-hydroxybenzophenone, 4-hydroxybenzophenone, 1-hydroxycyclohexyl phenyl ketone, 3,3′,4,4′-benzophenonetetracarboxylic dianhydride, 4-benzoylbiphenyl, diphenyl(2,4,6-trimethylbenzoyl)phosphine oxide/2-hydroxy-2-methylpropio-phenone, 2-hydroxy-4′-(2-hydroxy-ethoxy)-2-methylpropiophenone, 2,2-dimethoxy-2-phenylacetophenone, 2-methyl-4′-(methylthio)-2-morpholinopropiophenone, 2-hydroxy-4′-(2-hydroxyethoxy)-2-methylpropiophenone, 2-hydroxy-2-methylpropiophenone, 1-hydroxycyclohexyphenylketone, methylbenzoyl formate, diphenyl (2,4,6-trimethylbenzoyl)phosphineoxide, 2-benzyl-2-(dimethylamino)-4′-morpholinobutyrophenone, phosphine oxide, phenyl bis(2,4,6-trimethylbenzoyl), 2-benzyl-2-(dimethylamino)-4′-morpholinobutyrophenone, 2-methyl-1-[4-(methylthio)phenyl]-2-(4-morpholinyl)-1-propanone, 2-benzyl-2-(dimethylamino)-1-[4-(4-morpholinyl) phenyl]-1-butanone, 2-dimethylamino-2-(4-methylbenzyl)-1-(4-morpholin-4-ylphenyl)-butan-1-one, bis(.eta. 5-2,4-cylcopentadien-1-yl)-bis(2,6-difluoro-3-(1h-pyrrol-1-yl)-phenyl)titanium, 2-isopropylthioxanthone, 2-ethyl anthraquinone, 2,4-diethylthioxanthone, benzil dimethyl ketal, benzophenone, 4-chlorobenzophenone, methyl-2-benzoylbenzoate, 4-phenylbenzophenone, 4′5′-diphenyl-1,1′-biimidazole, 2,2′-bis(2-chlorophenyl)-4,4′,5,5′-tetraphenyl-1,2′-bi-imidazole, 2,2′,4-tris(2-chlorophenyl)-5-(3,4-dimethoxyp-enly)-4-phenoxy-2.′2′-dichloroacetophenone, ethyl-4-(dimethyl-amino)benzoate, 2-ethylhexyl-4-(dimethyl-amino)benzoate, isoamyl 4-(dimethylamino)benzoate, 4-(4′-methylphenylthio)-benzophenone, 4,4′bis(diethylamino) benzophenone (Michler's ethylketone), 1,7-bis(9-acridinyl)heptane, n-phenyl glycine, or combinations thereof.

In some embodiments, a chemical or thermal cross linker is used to encapsulate the luminescent dye and enzyme. Non-limiting embodiments of such crosslinkers include tetramethylethylenediamine (Temed), azobisisobutyronitrile (AIBN), persulfate, and the like.

### Enzymes

Examples of the enzyme/catalase molecules include, but are not limited to, glucose oxidase and lactate oxidase, lactate oxidative decarboxylase, lactic oxygenase, lactate oxygenase, lactic oxidase, L-lactate monooxygenase, lactate monooxygenase, L lactate-2-monooxygenase, cholesterol oxidase, alcohol oxidase, bilirubin oxidase, ascorbate oxidase, choline oxidase, pyruvate oxidase, sarcosine oxidase, tyramine oxidase, Acyl-CoA oxidase, Nicotinamide adenine dinucleotide (NADH, or NAD in a reduced state) oxidase, nicotinamide adenine dinucleotide phosphate (NADPH, or NADP in a reduced state) oxidase, cortisol enzymes, beta-hydroxybutyrate dehydrogenase. Additionally, co-factors may be needed to enable the enzymes to function. Thus, co-factors may be mixed with enzymes. Non-limiting embodiments of co-factors include nicotinamide adenine dinucleotide (NAD), flavin adenine dinucleotide (FAD), cobalamine, thiamine pyrophosphate, methylcobalamin, menaquinone, methanofuran, and lipoamine.

In some embodiments, the luminescence dye is tethered to a particle when in solution. The solution can be, but is not limited to, organic and inorganic solvents, ionic liquids as well as water. In some embodiments, the luminescent dye is tethered to a particle via ion interactions in which the particle is positively or negatively charged. In some embodiments, a particle is exposed to a chemical that positively or negatively charges the particle. The chemical for the surface modification can be due to exposure to the non-limiting examples of acids, bases, zwitterionic materials, plasma gas, and the like. In some embodiments, the particle surface is modified with chemical reagents. This chemical modification can result in functional groups located on the surface of the particle. These functional groups can result in the addition of, but not limited to, amine, hydroxyl, carboxylic acid, carbonyl, ester, aldehydes, n-hydroxysuccinimide, and carbodiimide groups located on the surface of the particle. In some embodiments, the chemically modified particle is chemically attached to the luminescence dye via a chemical reaction via said functional groups for immobilization.

In some embodiments, the luminescent dye is dissolved in a solution containing additives to immobilize the dye onto a substrate. A list of substrates has been previously mentioned. In a version of this embodiment, the additive immobilizes the luminescence dye on the substrate as the solvent of the solution dissolves. The additives can be, but are not limited to, polystyrene, Low-Density Polyethylene (LDPE), Polypropylene (PP), polycarbonate, polylactide, acrylic, acrylonitrile butadiene, styrene. For example, in one non-limiting example, PtTPTBP, chloroform, and polystyrene are mixed. Upon exposing PTFE with this mixture, chloroform evaporates leaving PtTPTBP immobilized within a polystyrene network.

In some embodiments, the sensing matrices are adhered onto the sensor using an adhesive. The adhesive precursors, or non-adhesive state, can be gaseous, liquid, or solid. Moreover, in a non-limiting fashion the adhesive may solidify/cure by light exposure, chemical exposure, temperature, humidity, or time. Non-limiting embodiments of the adhesives are Loctite 4981, Loctite 4541, Loctite 401, Loctite 404, NOA 87, NOA 65, NOA 68TH, NOA 73, NOA 75, NOA 88, Original Gorilla Glue, Gorilla Clear Multipurpose Adhesive, EPO-TEK E4110, EPO-TEK H20E, EPO-TEK H22, EPO-TEK H31, Structalit 701, Vitralit 1655, Vitralit 4731, Vitralit 6108, Vitralit 7989, Cyanolit 203 TX, Cyanolit 732 F, Electolit 323, Permabond 4C10, Nusil MED2-4213, Nusil MED2-4013, Nusil MED2-4420, Nusil MED2-161. Non-limiting materials found in these components include cyanoacrylate, silicon, diphenylmethane diisocyanate, isoprene, butyl isoprene, nitrile isoprene, styrene butyl isoprene, butadiene isoprene, polyurethane, acrylic, methacrylic acids, epoxies, polyether polyols, polychloroprene, styrene, polyisobutylene, polysulfide, polyamide, styrene-acrylic copolymer, styrene-acrylic emulsion polymer, styrene butadiene, resin and hardeners, or a combination of these materials.

### Data Acquisition

In one embodiment, the data acquisition electronics and/or circuitry are contained within the circuitry located on the recording unit, while in other embodiments, the data acquisition system is located on a separate chip. In some embodiments, the data acquisition electronics and/or circuitry is from the non-limiting list of a microcontroller and controller packages such as, such as the PJRC Teensy series, raspberry pie, arduino UNO, NodeMCU, MSP430 Launchpad, and the STM32. In another embodiment, the data acquisition electronics/circuitry includes wireless signal transmission, such as, but not limited to, Bluetooth. Wireless transmission can be received by a device such as a hand-held unit or a smartphone, tablet computer, laptop, or equivalent technology. In some embodiments, the sensor has an alarm to warn about measured analyte concentrations.

### EXAMPLE

The following is a non-limiting example of the present invention. It is to be understood that said example is not intended to limit the present invention in any way. Equivalents or substitutes are within the scope of the present invention.

Filter-Coated Photodetector: For the pH sensor, silicon photodetectors (302) were coated with an optical filter as follows. A plastic green bandpass filter (Primary Green Filter, Lee filters, USA) was first cut into a 2 cm×2 cm square. The square filter was then placed onto the surface of a 2 mm×1.25 mm silicon photodetector (302) (SFH2716, OSRAM Opto Semiconductors, Germany) that had been mounted on a microscope glass slide. The edges of the filter were held in place by two additional glass slides. A Varitemp VT-750C heat gun (Master Appliance, USA) at a temperature setting of 250°° C. was then used to melt the plastic green bandpass filter onto the photodetector (302). An example of said filter-coated photodetector is shown in FIG. 11.

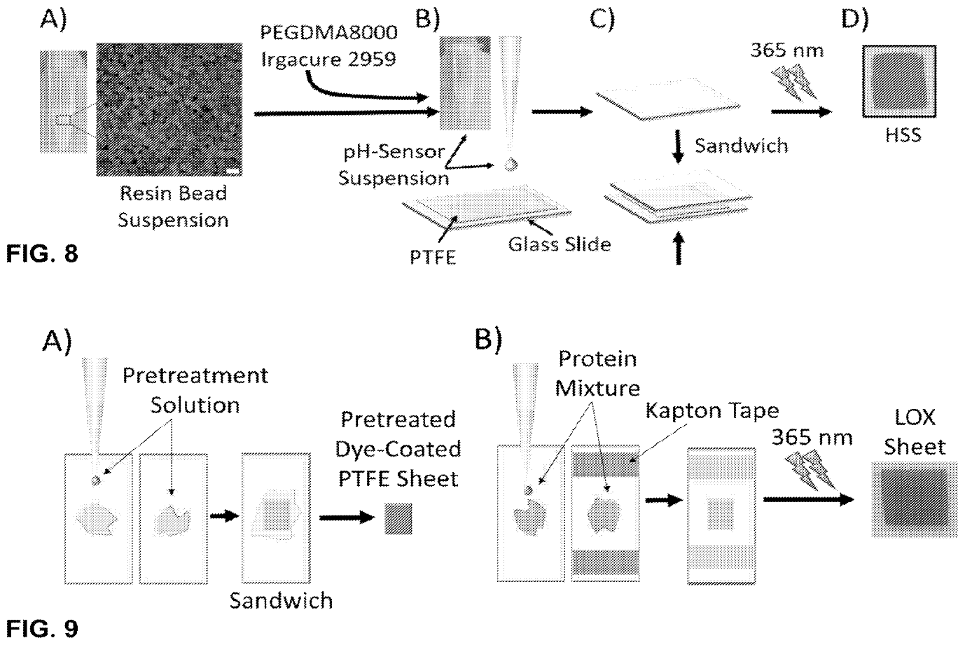

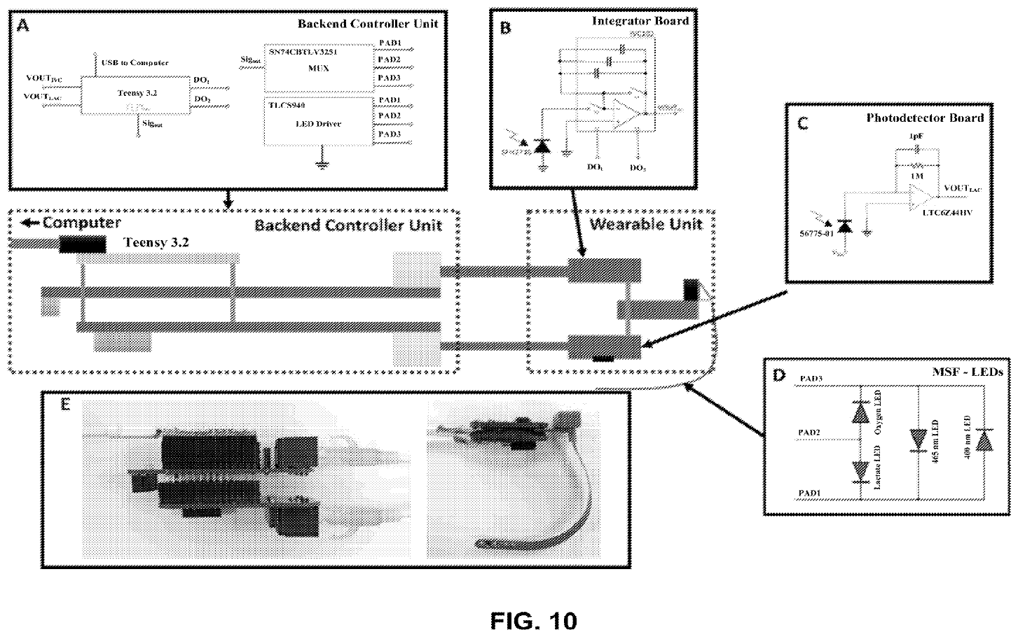

pH Sensor Sheet (HSS): First, a 12.7 mM 8-Hydroxypyrene-1,3,6-trisulfonic acid trisodium salt (HPTS) stock solution was formulated by dissolving 99 mg of HPTS (MilliporeSigma, USA) in 15 mL of Milli-Q water (MilliporeSigma, 18.2 Mohm·cm at 25° C.). 10 g of 45-150 μm diameter Dowex® 1X8 resin beads (MilliporeSigma, USA) were then suspended in the stock solution within a 20 mL disposable scintillation vial. This process yields the resin bead suspension and allows the negatively charged sulfonate groups on HPTS to ionically bind to the positively charged Dowex Resin. 500 μL of the resin bead suspension was then added to a 1.5 mL amber glass vial along with 50 mg of poly(ethylene glycol) dimethacrylate 8000 (PEGDMA8000, Polysciences, USA), and 12 mg of 2-Hydroxy-1-(4-(2-hydroxyethoxy) phenyl)-2-methylpropan-1-one (Irgacure 2959, Sigma-Aldrich, USA), creating the pH-sensor suspension. To fabricate the pH Sensor Sheet (HSS) 304, 305, a thin circular sheet of hydrophilic polytetrafluoroethylene (PTFE, H050A047A, 35 μm thick, 0.50 μm pores, 47 mm diameter, Sterlitech, USA) was cut into a 1.2 cm×1 cm rectangle and placed onto a microscope glass slide. 40 μL of the pH-sensor suspension was then pipetted onto the cut PTFE sheet. The sheet was then sandwiched between two glass slides until the pH-sensor suspension uniformly coated one side of the sheet. The pores of the PTFE sheet are two orders of magnitude smaller than the resin bead diameter, preventing resin penetration into the sheet. The coated sheet was then polymerized for 15 min using 365 nm wavelength light emitted from an 8-watt dual-ultraviolet (UV) transilluminator (VWR, USA) to produce the HSS. FIG. 8. The HSS was retrieved with tweezers and left to swell for at least 2 hrs in Milli-Q water within a 20 mL scintillation vial.

### Oxygen-Sensitive Dye-Coated PTFE Sheet Fabrication:

Oxygen-sensitive dye solution was created by mixing 4 mg of PtTPTBP dye (Frontier, USA), 60 mg of polystyrene (molecular weight: 2500, Sigma-Aldrich, USA) and 900 μL of chloroform (Sigma-Aldrich, USA) in a 1.5 mL amber vial. The resulting concentrations are 440.873 μM PtTPTBP and 0.024 mM polystyrene in 900 μL chloroform. 200 μL of this solution was pipetted onto a circular PTFE sheet. The chloroform was allowed to evaporate resulting in the oxygen-sensitive dye-coated PTFE sheet. This dye-coated PTFE sheet served as the base of both the Lactate Oxidase (LOX) and Oxygen Sheets.

Solutions for Preparing the LOX Sheet: The LOX sheet is formulated using two solutions: (1) Protein Mixture and (2) Pretreatment Solution. Protein Mixture is composed of 0.060 mM LOX (LCO-301, 108 U/mg, 0.0096 mg/μL, Toyobo, Japan), 193.13 mM poly(ethylene glycol) dimethacrylate 2000 (PEG DMA 2000, Sigma-Aldrich, 0.386 mg/μL, USA), 43.33 mM Lithium phenyl-2,4,6-trimethylbenzoylphosphinate (LAP, 0.0127 mg/μL, Sigma-Aldrich, USA), 1.16 mM catalase (AN366A, 5410 U/mg, 0.28 mg/μL, BBI Solutions, U.K.), and 1× Phosphate Buffered Saline Solution (1× PBS). Pretreatment Solution is composed of 75% v/v poly(ethylene glycol) diacrylate 400 (PEGDA400, Polysciences, USA), 25% v/v Milli-Q water, and 28 mM LAP (0.00825 mg/μL).

LOX and Oxygen Sensor Sheets: LOX Sheets (304) were fabricated from oxygen-sensitive dye-coated PTFE sheets, the Protein Mixture containing enzymes LOX and catalase, and the Pretreatment Solution. First, a 1 cm×1 cm square of the dye-coated PTFE sheet was excised using a razor blade. 4.5 μL of Pretreatment Solution was then pipetted onto each of two microscope glass slides. After, the dye-coated PTFE sheet was sandwiched between the two glass slides to force the Pretreatment Solution into the pores of the sheet. The purpose of the Pretreatment Solution is to establish a porous network within the dye-coated PTFE sheet that limits the diffusion of lactate and oxygen. Next, two parallel doubled-layered strips of 25 μm-thick Kapton tape (Tapes Master, USA) were applied to a glass slide to function as spacers. 4.5 μL of Protein Mixture was then pipetted in between the spacers and onto a second slide. The pretreated dye-coated PTFE sheet was then placed between the spacers and sandwiched between the glass slides. While sandwiched, the sensor sheet (304) was polymerized with the 8-watt dual-UV transilluminator for 5 min to yield the LOX Sheet. FIG. 9. The Oxygen Sheet (304) was fabricated in similar fashion except for the exclusion of LOX and catalase in its Protein Mixture.

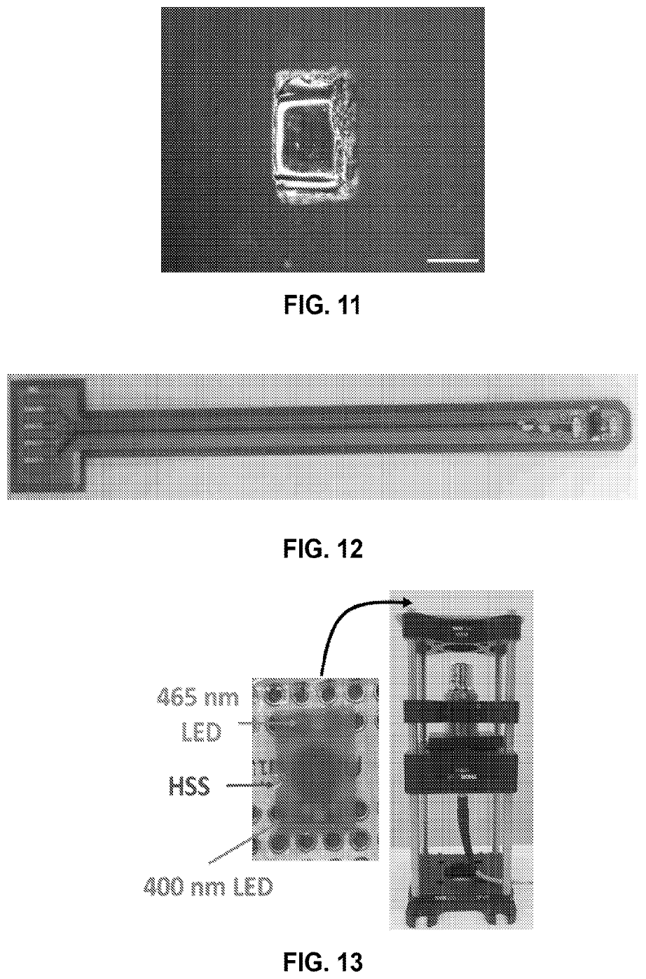

PALS Circuitry: PALS consists of custom-made printed circuit boards (fabricated by OSH Park, USA) designed using Eagle (Autodesk, USA). These custom boards were manually assembled and make up the three main subunits of PALS. FIG. 8. The first subunit is the flexible sensor (102), FIG. 12. The second subunit is the recording unit comprising three circuit boards: (1) the integrator board for pH sensing based on the IVC102 transimpedance amplifier (Texas Instruments, USA)., (2) the photodetector board for lactate and oxygen sensing, and (3) the connector board to connect the photodetector and integrator boards to the flexible sensor (102). The last subunit is the backend controller unit comprising a microcontroller Teensy 3.2 (PJRC, USA) and two circuit boards: (1) the controller board for charlieplexing and tuning the drive current of the flexible sensor LEDs and (2) the IVC power supply board. Custom software was written for Teensy 3.2 to charlieplex the LEDs and acquire data, using a combination of Arduino (Arduino, USA) with Teensyduino library (PJRC, USA) and LabView (National Instruments, USA). Custom designed housings were printed by a stereolithography printer (Prusa SL1, Prusa Research, Czech Republic) to protect and house the recording unit (103). The housing unit serves to also pressure-connect the prongs of the 5-spring battery connector (009155005852006, AVX Corporation, USA) on the connector board to the gold pads of the flexible sensor (102).

Flexible Sensor Fabrication: The flexible sensor FIG. 2, (102) contains optoelectronic components for both pH and lactate sensing. The optoelectronic components for pH sensing are a 400 nm light emitting diode (LED) (SM0603UV-400, Bivar, USA), a 465 nm LED (APT1608QBC/G, Kingbright, USA) and a filter-coated photodetector (302). Two 625 nm LEDs (APHHS1005LSECK/J3-PF, Kingbright, USA) are used for lactate and oxygen sensing. The flexible sensor (102) was coated with Loctite EA E-60NC (1:1 resin to hardener mix ratio, Henkel, Germany) for waterproofing. The waterproof coating was left to cure overnight.

### Sensor Film Application:

The HSS was the first sensor sheet applied. After swelling, the HSS was removed from Milli-Q water and placed onto a glass slide. A razor blade was used to remove hydrogel from the sheet edges to expose underlying PTFE. This process resulted in a rectangular hydrogel with dimensions of roughly 1 cm×0.8 cm. Loctite 4981 was then applied along the width of the flexible sensor (102), between the 465 nm LED and the top-most 625 nm LED. The shorter side of the HSS (0.8 cm) with exposed PTFE was laid down onto the Loctite 4981, with the resin-tethered HPTS side facing the LEDs. The Loctite 4981 (Henkel, Germany) was then allowed to cure. The flexible sensor (102) was then flipped over, and Loctite 4981 was applied to the top edge and along the lateral edges of the back of the flexible sensor (102). The remaining exposed PTFE edges of the HSS were then pressed in contact with the Loctite 4981 and allowed to cure.

After HSS was cured onto the flexible sensor (102), LOX and Oxygen Sheets were applied onto the flexible sensor (102). The sheets were cut into smaller sheets (1.8 mm×1.5 mm) immediately following polymerization and adhered directly onto the 625 nm LEDs with Loctite 4981. When all sensing sheets were applied, the flexible sensor (102) was allowed to incubate in 1× PBS for at least 8 h at room temperature prior to testing.

### PALS pH Sensor Testing Method:

pH test solutions were prepared following Sigma-Aldrich's Phosphate Buffer Preparation Table, using potassium phosphate monobasic anhydrous (795488-500G, Sigma-Aldrich, USA), sodium phosphate dibasic heptahydrate (S9390-1 KG, Sigma-Aldrich, USA), and Milli-Q water. Polynomial equations were fitted onto both reagent quantities listed in the Preparation Table to interpolate pH solution formulations. The pH solutions were probed using a Mettler Toledo FiveEasy pH probe (Sigma Aldrich, USA). pH probe calibrations were completed with pH 4.01, 7.00, and 10.01 buffer solutions (Orion™ Standard All-in-One™ pH Buffer Kit, 910199, ThermoFisher Scientific). Only calibrations with slopes greater than 95 were used. For pH testing in vitro, measurements were obtained at room temperature. For PALS pH testing in vitro, each flexible sensor (102) was placed in a 20 mL scintillation vial and incubated in the series of pH solutions. For testing, except for rise times studies, the flexible sensor (102) was first washed with the pH test solution 10 times and then allowed to equilibrate for 30 s. After measurements were obtained, the test solution was aspirated. This process was completed for each new test solution. For rise time studies, only 1 wash was completed when introducing the next test solution.

### PALS Lactate Sensor Testing Method:

Lactate test solutions were prepared with L-(+)-Lactic acid solution (27714-1L, Sigma Aldrich, USA) and 1× PBS. pH adjustments were made with 0.1 M HCl and 0.1 M NaOH (43617, Sigma-Aldrich). 0.1 M HCl was formulated by diluting 11 N HCl (A144C-212, Fisher Scientific, USA) with Milli-Q water. Unless otherwise noted, pH of lactate solutions was adjusted to 7.45. The lactate solutions were verified using a YSI 2300 STAT Plus Glucose and Lactate Analyzer (Yellow Springs Instrument, USA). For lactate testing in vitro, measurements were obtained at room temperature. Each flexible sensor (102) was placed in a 60 mm plastic dish (BD Falcon, USA) and incubated in the series of lactate solutions. For testing, except for rise times studies, the flexible sensor (102) was first washed with the lactate test solution 10 times and then allowed to equilibrate for 30 s. After measurements were obtained, the test solution was aspirated. This process was completed for each new test solution. For rise time studies, only 1 wash was completed when introducing the next test solution.

Benchtop Optical System Set-up: This setup was used in the development of the invention of this application and was not intended to be part of the medical device. The benchtop optical system (FIG. 13) was built using ThorLabs (USA) assembly rods (SR05, SR1, and SR1.5 rods) with vertical cage system mounting plate CPVM (ThorLabs, USA) serving as the base component. Building from bottom to top, cage plate LCP01T (ThorLabs, USA) was suspended above the CPVM to add on a jacketed multi-mode optical fiber patch cable (0.22 NA, 200 μm core, M92L02, ThorLabs, USA) coupled to a collimator (F810APC-543, ThorLabs, USA). The opposite end of the optical fiber patch cable was connected to a CCS200 spectrometer (ThorLabs, USA). Above the collimator, a LCP02 (ThorLabs, USA) cage plate adapter housing a 5× objective lens (0.10 NA, Newport, USA) was added to capture HSS-emitted light Settled above the objective lens on an additional LCP02 was a Perma-Proto board (Adafruit, USA) with 400 and 465 nm dominant-wavelength LEDs soldered on opposite ends of a drilled hole (diameter=1/inch). In parallel, exposed PTFE of an HSS was used to adhere the sheet onto a μ-Slide 2 well (Ibidi, USA) using Loctite 4981. The adhesive was allowed to dry for at least 5 min prior to testing. The well was secured above the protoboard using tape. The well was positioned, such that upon testing, the HSS sat over the LEDs and emitted light was collected through the drilled hole with the 5× objective lens.

ThorLabs Optical Spectrum Analyzer (OSA) software was used to acquire spectra data. An Arduino Uno with custom Arduino software (Arduino, USA) was used to power the LEDs (5V output). Resistors (+5% tolerance range, BOJACK, China) with total resistance values of 1450 and 830 ohms were placed in series with the 400 and 465 nm LEDS, respectively. Integration time for both LEDs was 1 s. For pH testing, the HSS was first washed with the first pH test solution 10 times and then allowed to equilibrate for 30 s. 3 emission spectra were recorded for each LED and averaged after background spectrum (no LED excitation) subtraction. The background-subtracted emission spectrum was then saved as a text file. To exchange pH solution, the solution previously tested was aspirated under vacuum and the next pH test solution was tested as previously mentioned. MATLAB (MathWorks, USA) was used for subsequent analysis.

LED Spectra Acquisition System: To acquire LED spectra, a caged system containing a suspended optical fiber was created. All items were procured from ThorLabs (USA). Blank cage plate (LCP03) was used as the base of the system. Assembly rods SR05, SR1, and SR1.5 rods were used to build vertically. Vertical mount plate CPVM threaded with a SM1SMA fiber adapter was used to incorporate jacketed fiber patch cable M92L01 into the system. The distance between the M92L01 orifice and the blank cage plate was approximately 90 mm. The opposite end of the jacketed optical fiber was connected to a CCS200 spectrometer. MSF with LEDs were taped down and placed in the center of the blank cage plate such that upon LED activation, M92L01 would capture emitted light. Thorlabs OSA software was used to acquire spectra. Unless otherwise stated, integration times were set to 100 ms. To acquire data, LabView code (National Instruments, USA) was used to turn on the MSF LEDs and spectra were then obtained using the LED Spectra Acquisition System. Drive currents for each LED were less than 10 mA.

Operational Parameters: For pH sensing, LED currents were set to 6 mA and HSS-emitted light was sampled at 10 Khz. Detection integration times (<1s) were tuned at the start of each experiment. The tuning occurs in the first test solution of each in vitro experiment or following insertion for the in vivo experiment. Specifically, integration time was tuned such that the IVC hold voltage ranged between 1-2 V, corresponding to 30-61% of the total integration capacitance. For lactate sensing, each of the 625 nm LEDs were illuminated one at a time. The LEDs were driven by a waveform comprising 21 cycles of a square wave with peak current of 9 mA, at a frequency of 5 kHz, and 25% duty cycle. PtTPTBP emission was sampled simultaneously at 500 kHz.

Statistical Analysis: Measurements are reported as the mean and standard error of the mean. Statistical analysis included a one-way analysis of variance (ANOVA) with Tukey post-hoc comparison or coefficient of variance (CV). p<0.05 denotes statistical difference. Prism 8 (GraphPad, USA) was used for statistical analysis.

In Vivo Rabbit Study: The PALS implant study was conducted with the approval of the Institutional Animal Care and Use Committee at the University of California Irvine. A New Zealand White Rabbit was sedated with a 2:1 ratio of Ketamine Hydrochloride (100 mg/ml, Ketaject, Phoenix Pharmaceutical Inc., USA): Xylazine (20 mg/ml, Anased, Lloyd Laboratories) at a dose of 37.5 mg/kg of Ketamine and 5 mg/kg of Xylazine IM using a 25-gauge ⅝-inch needle. The mixture of keta-mine and xylazine was infused via the animal's right marginal ear vein. The animal was intubated and placed on mechanical ventilation with a tidal volume of 50 mL per breath, respiratory rate of 20 breaths/min, and 100% oxygen. An arterial catheter was placed within the right femoral artery for systemic blood pressure measurements and arterial blood gas sampling. To implant the flexible sensor (102) in the subcutaneous space of the inner left thigh of the rabbit, first, an incision (length=0.8 cm) was created with a scalpel. Next, a Metzenbaum dissecting scissor (Cole-Parmer, USA) was inserted inside the incision to separate skin from underlying muscle, to accommodate the tip of the flexible sensor (102). After flexible sensor (102) tip insertion, Loctite 4981 adhesive (Henkel, Germany) was applied at the incision site to adhere the flexible sensor (102) onto the skin and seal the incision. The PALS recording unit (103) was then placed on the skin and aligned to the flexible sensor tip 102. Hypafix adhesive (USA) was placed over the PALS recording unit (103) to limit sensor movement. After baseline measurements, the PALS was disconnected, and the animal was placed inside a sealed chamber which was then moved into a fume hood. The animal received 800 ppm chlorine gas (Airgas, USA) for 6 min followed by a 5 min rest period. 1 mL of 100 mM trihistidyl cobinamide was then administered through the right marginal ear vein. The animal was returned to the surgical room and the PALS was reconnected to resume monitoring. At the conclusion of the study, the animal was euthanized per standard procedures (1 mL of Euthasol, Virbac, USA).

Analysis of In Vivo Data: During the study, blood was drawn from the right femoral artery at 8 time points. Four blood draws were retrieved before and after chlorine gas and cobinamide infusion. Blood pH and lactate concentration was immediately assessed with an i-STAT (Abbott Laboratories, USA) using Abbott CG4+ cartridges (Abbott Laboratories, USA). PALS pH and lactate measurements were retrospectively calibrated to the blood pH and lactate concentrations obtained from the i-STAT with a linear regression model.

### The PALS pH Sensor

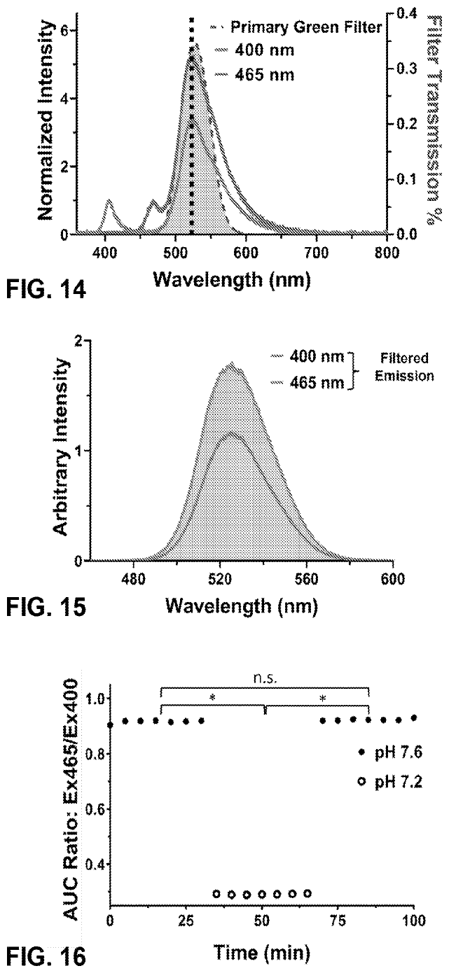

pH Sensing by Dual LED Excitation of the HSS and Single Band Detection: The PALS uses a dual LED excitation, single band photodetection scheme for measuring pH. To enable such sensing, the tip of the flexible sensor (102) comprises two surface-mount LEDs to excite an HSS and a coated photodetector (302) to collect pH-sensitive emissions. To first determine if this is a viable scheme, a benchtop optical system was constructed (FIG. 13). This system includes surface mount 400 nm and 465 nm dominant-wavelength LEDs soldered onto a protoboard, and a microscope objective lens that couples emitted light to a spectrometer (FIG. 13). pH-sensitive spectra from an HSS in a pH 7.6 solution were collected with serial illumination from the two LEDs (FIG. 14). HSS emission spectrum ranges from 470 nm to 660 nm and has a peak value at 520 nm. While the 400 nm LED light does not overlap the HSS emission spectrum, the 465 nm LED does, necessitating an optical filter that mitigates the effects of this spectral crosstalk. Ideally, such a filter should transmit the HSS emission spectrum (FIG. 14) and be easily applied onto the surface of a photodetector (302). The Primary Green Filter was selected which can be applied onto a photodetector (302). To simulate the filtering behavior of Primary Green Filter, its transmission spectra was multiplied with HSS spectra (one per LED). The resulting filtered-spectra show that the 465 nm LED light has a negligible contribution to the fluorescence signal (FIG. 15). To test for pH sensitivity and reversibility, HSS spectra in solutions having pH 7.2 or 7.6 were collected and then multiplied by the Primary Green Filter transmission curve. The ratio of area-under-the-curve (AUC Ratio: _{Ex465}/_{Ex400}) was determined at these two pH values. Results indicate that the dual LED, single-band detection scheme is stable and reversible, and can be utilized as a pH optode at the tip of the flexible sensor (102) (FIG. 16).

The pH Optode: The pH optode comprises three of the optoelectronic elements at the tip of the PALS flexible sensor (102), FIG. 12. The optode is fabricated by placing an HSS at the tip of the flexible sensor (102) such that it overlaps the two LEDs and filter-coated photodetector (302). HPTS' peak emission intensity at 520 nm reflects its absorption property where HPTS absorbs light more efficiently at 450 and 405 nm at high and low pH's, respectively. Therefore, after dual light excitation at 405 and 450 nm, HPTS can reliably measure pH through a ratiometric analysis at its peak emission wavelength. Photocurrents from the photodetector (302) are amplified and converted to a voltage using a transimpedance amplifier. pH is related to the ratio of the voltages acquired with 465 nm and 400 nm LED illumination (R_{Ex465}/_{Ex400}) after background subtraction (no LED on).

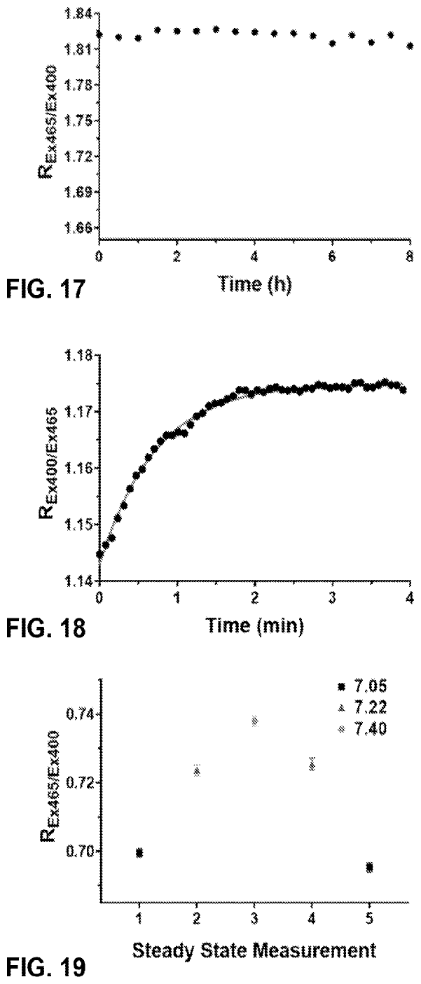

pH Sensing In Vitro: To determine baseline pH sensor stability, measurements were obtained every 30 min for 8 h in pH 7.45 solution. Unless otherwise stated, each reported steady state measurement is an average of 20 repeats. No signal drift was detected (FIG. 17; CV=0.002). The stability is due in part to the quality of our custom LED current control circuitry (provided by the TLC driver) that produces consistent LED output power. pH sensing rise time was assessed by first incubating a sensor in a solution with pH 7.45 and then exchanging for a solution with pH 7.01. R_{Ex400}/_{Ex465} Was measured every 4.8 s following media exchange. Dynamics are described by a rising exponential plateau model (R²=0.99, standard error of the estimate (S_{y.x})=0.006) with a rise time of 2.63 min (FIG. 18). This rise time is short enough to capture critical physiological events such as subdermal scalp acidosis following fetal tachycardia in high-risk births, which occurs on the time scale of minutes.

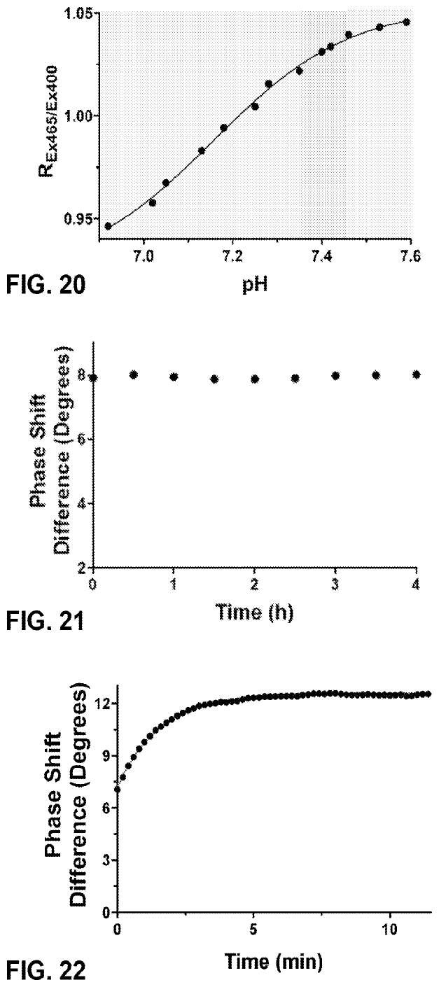

pH sensing reversibility was assessed by sequential measurements of solutions having pH of 7.05, 7.22, and 7.40 (FIG. 19). One-way ANOVA indicates a significant effect across groups (p<<0.01). Tukey post-hoc comparison with adjusted p-values shows significant differences between the three unique pH solutions (p<<0.01 for each comparison) but no significant differences between repeated pH solutions (p>0.25 for each comparison). pH sensing sensitivity and range was assessed by exposure to thirteen solutions, with pH ranging from 6.92 to 7.59 (FIG. 20). Data follows a Boltzmann sigmoidal model (R²>0.99, S_{y.x}=0.002) with a pKa of 7.16, which is similar to the pKa of the HPTS dye alone (FIG. 20). Importantly, wellness-of-fit indicates PALS will have clinical relevance across the pathophysiological range including acidosis and alkalosis.

### The PALS Lactate Sensor

Lactate Optode: In brief, two oxygen-sensitive PtTPTBP dye sheets are mounted on two 625 nm dominant-wavelength LEDs (FIG. 12). One of these sheets also contains the enzyme LOX. The working optode has the enzyme-containing sheet, whereas the reference optode has the enzyme-free sheet. The LEDs are illuminated in sequence, and the oxygen-sensitive emitted light is detected by a photodetector (303) within the PALS recording unit (103). Lactate is related to the phase shift difference between the working and reference optode signals. The phase shift is defined as the phase difference (assessed by Fourier Transform) between the LED drive-current waveform and corresponding photodetector (303) signal at the drive frequency.

Lactate Sensing In Vitro: To determine baseline lactate sensor stability, measurements were obtained every 30 min for 4 h in 4 mM lactate solution (FIG. 21). Unless otherwise stated, each reported steady state value is the mean of 10 measurements. No significant signal drift was detected (CV=0.007). The stability is due in part to the inclusion of the enzyme catalase that scavenges hydrogen peroxide, known to degrade proteins. Lactate sensing rise time was assessed by changing the test media from 0 mM (1× Phosphate Buffered Saline Solution, PBS) to 6 mM lactate. Phase shift difference measurements were recorded every 12 s following media exchange. Signals follow an exponential plateau model (R²=0.99, S_{y.x}=0.06) with a rise time of 4.8 min (FIG. 22). This rise time is comparable to commercial continuous glucose monitors such as the Dexcom G6 and Medtronic Guardian, which are reported to have an average rise time in vivo of 9.5 min and are effective in guiding insulin therapy.

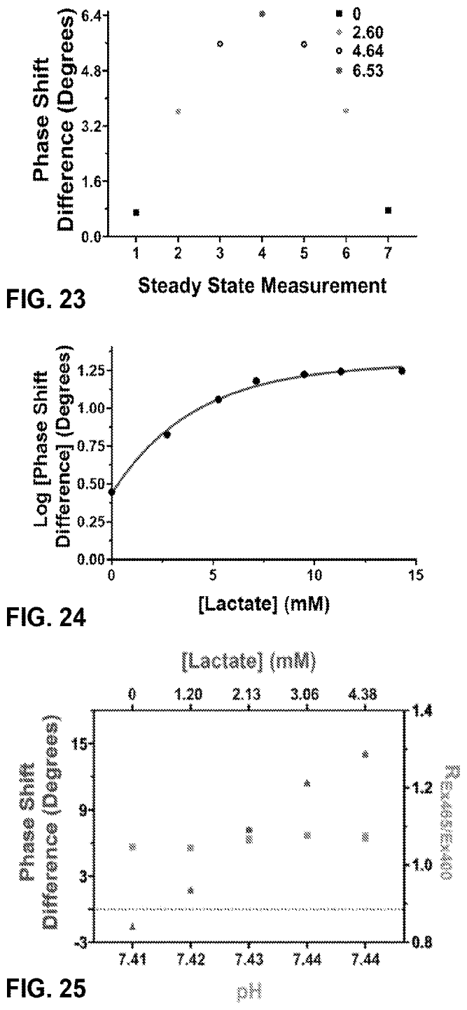

Lactate sensing reversibility was evaluated by cycling lactate solutions having concentrations of 0, 2.60, 4.64, and 6.53 mM. One-way ANOVA detected differences between groups (p<<0.01) (FIG. 23). Tukey post-hoc comparison with adjusted p-values shows no significant differences between the pairing of identical lactate solutions (p>0.87), while significant differences were found between solutions of different lactate concentrations (p<<0.01). Lactate sensitivity and range were evaluated by incubation in seven lactate solutions ranging in concentration from 0 to 14 mM. Data follows an exponential plateau model (R²=0.99, S_{y.x}=0.03), showing sensitivity to lactate from 0 to 9 mM (FIG. 24). These results show PALS lactate sensing can distinguish between concentrations within the pathophysiological range.

### PALS Multi-Analyte Sensing

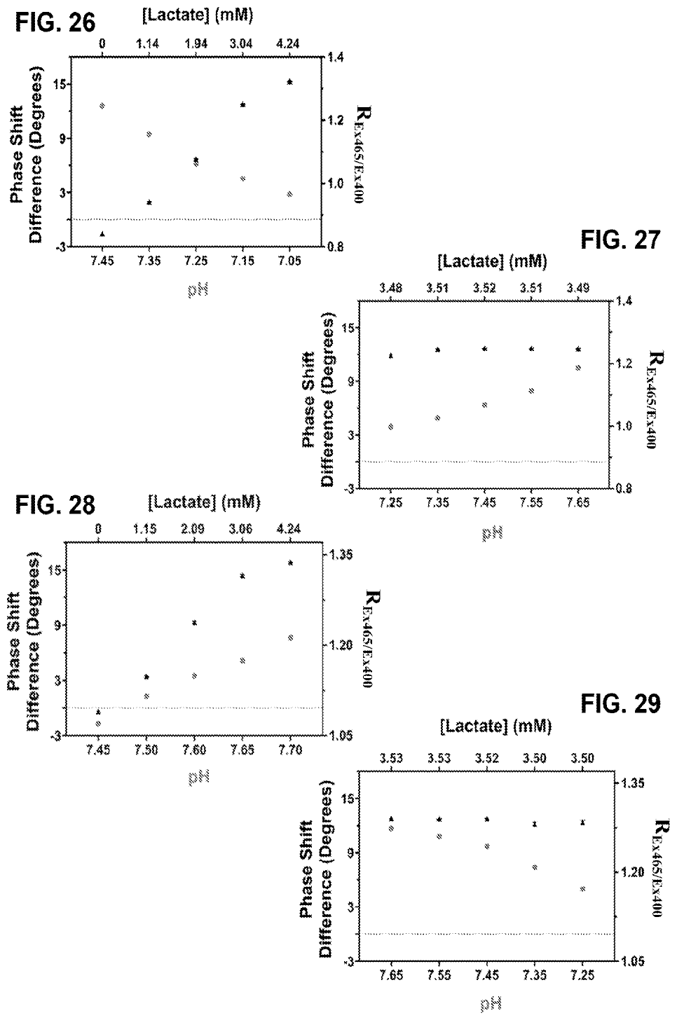

Combined PALS pH and lactate sensing (FIG. 7) was tested in in vitro models of five pathophysiological conditions. In a model of hyperlactatemia, pH was maintained at 7.43±0.02 while lactate was increased. PALS signals report the increase in lactate while the pH signals did not significantly change (CV=0.018, FIG. 25). In a model of lactic acidosis, as experienced in sepsis, solutions were formulated to have a decrease in pH and an increase in lactate, and PALS successfully reported these changes (FIG. 26). In a model of extreme metabolic alkalosis, as can occur with chronic vomiting and diarrhea, pH was increased while lactate was held at 3.50 mM±0.02.46 PALS signals report the increase in pH and constant lactate concentration (CV=0.027, FIG. 27). In a model of panic-disorder patients that exhibit respiratory alkalosis and hyperlactatemia, solutions were formulated to have an increase in both pH and lactate concentrations. PALS signals successfully reflect these increases (FIG. 28). In a model of respiratory acidosis (as seen in chronic obstructive pulmonary disease) pH was formulated to decrease while lactate levels were held constant at 3.52±0.02.47 PALS signals report the decrease in pH and constant lactate concentrations (CV=0.019, FIG. 29). Collectively, these figures demonstrate PALS does not exhibit sensing modality crosstalk and can report physiologically relevant pH and lactate changes, which may aid clinicians in their practice of analyte-guided treatment towards improving patient outcome.

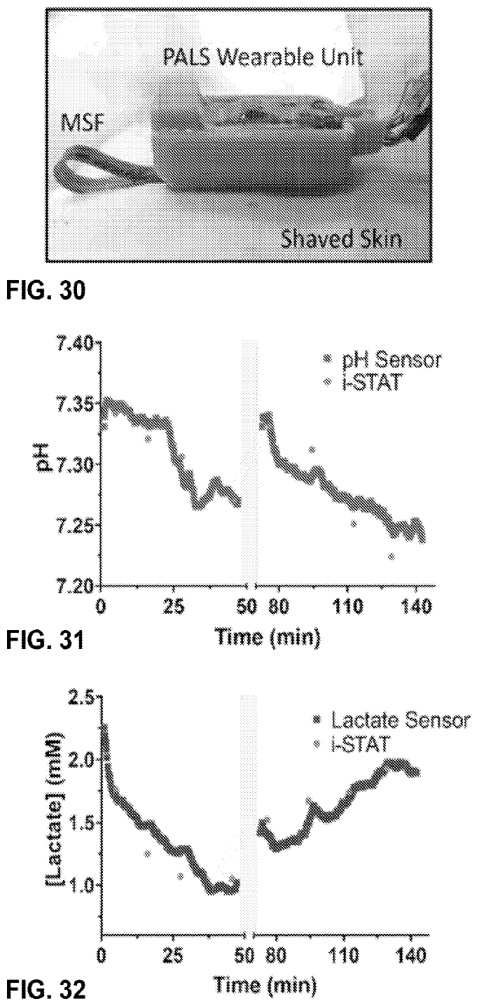

Multi-analyte Sensing In Vivo: PALS multi-analyte sensing was tested in an in vivo rabbit model of chlorine gas poisoning and cobinamide treatment. When chlorine gas reacts with water in the lungs, hydrochloric acid and hypochlorous acid are produced. The production of these acids damages the respiratory mucus membrane resulting in pulmonary edema and hypoxemia. PALS should therefore detect decreasing pH and increasing lactate values following chlorine gas administration. First, the flexible sensor (102) tip was implanted in the rabbit (FIG. 30). The results show that prior to drug infusion but early into intubation, PALS detected a decrease in both pH and lactate concentration, which agrees with the intermittent i-STAT blood assays (FIG. 31). After poisoning and cobinamide treatment, PALS detected the expected increase in lactate concentration and decrease in blood pH, which were also in agreement with the intermittent i-STAT blood assays (FIG. 32). Area-under-the-concentration time curve ratio between PALS and i-STAT measurements was calculated and assessed following FDA guidelines. Area-under-the-concentration time curve ratio for pH (0.979) and lactate (1.036) indicate PALS bioequivalence to the i-STAT blood analyzer in this study. Results demonstrate PALS reports clinically relevant pH and lactate changes, and at a higher frequency than can be offered by blood analysis alone.

Data for Photodetection on the Flexible Sensor: This example demonstrates the use of a Light emitting diode (LED) as a photodetector on the flexible circuit intended for implantation and measurement of body chemistries.

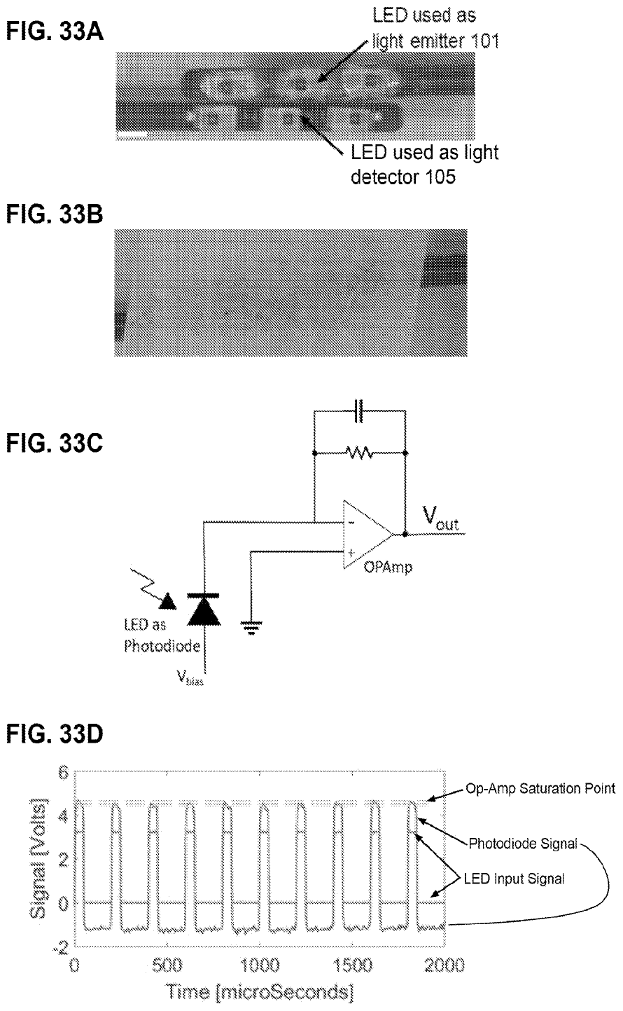

FIG. 33A shows two 0.6 mm-wide flex circuits, each having three attached LEDs. The two flex circuits were placed in contact with one another. Light produced by one LED on the first flex circuit was measured using an LED on the second flex circuit. One of the two flex circuits (top) was wired to supply a drive current to an LED, so that the LED emits light. The other of the two flex circuits (bottom) was wired so that one of the LEDs acted as a photodiode, or photodetector. Referring to FIG. 33B, to simulate light transmission through a diffuse medium, a polytetrafluoroethylene (PTFE) sheet was placed over the emission-LED/detection-LED pair. The LED acting as a photodiode is wired to a transimpedance amplifier, which may in turn be part of a circuit, with one example shown in FIG. 33C. Such a circuit can be used to amplify the signal and convert the photocurrent to a related photovoltage. This example circuit was originally designed for a standard photodiode. This methodology allows for photodiode-compatible acquisition circuit architectures to be applied either on the backend or front end of the circuit, such as reverse biasing or other alternate signal conditioning methods.

Importantly, the proof of concept experiment shown in FIG. 33A can be embodied on a single flex circuit or multiple flex circuits. The non-limiting embodiment in which both light emitting and light detecting LEDs are on a single flex circuit is preferred in this invention. However each element is not limited to only light emission or light detection. Functionality can be toggled. FIG. 33D shows a time-matched graph of the emission-LED input (or drive) signal (labeled “LED Input Signal”) and detection-LED (labeled “Photodiode Signal”) response measured by an oscilloscope (Keysight) using the configuration in FIG. 33A. The photodiode signal has been median filtered with a window of five points to remove shot noise. The photodiode signal peak amplitude approached the operational-amplifier saturation voltage, which indicates the photodetector can measure light intensity in the anticipated intensity range. The circuit and LED selection can be reconfigured to allow for detection of a porphyrin dye signal, or other static or dynamic signal.

In some embodiments, LEDs on the same flex circuit can be used exclusively as light emitters or light detectors. In other embodiments, LEDs on the same flex circuit can be used as either light emitters or photodetectors in this manner.

Without wishing to limit the invention to a particular theory or mechanism, PALS can have a major impact on patient outcomes in conditions such as sepsis and organ failure where attentive monitoring of pH and lactate are known to improve outcomes. Importantly, the core elements of PALS can be replicated and modified to sense additional analytes on the flexible sensor (102). For example, similar to the operating principle of HPTS, fluorescent dyes Fura Red AM and SBFI-AM exhibit a change in their absorbance behavior based upon the concentration of calcium and sodium, respectively. Consequently, the dual-excitation, single band detection scheme could be readily employed to continuously monitor these analytes. Moreover, the lactate sensing scheme is generalizable to additional oxidases and its corresponding analyte including those for glucose and alcohol. The addition of these analytes can broaden the applicability of the flexible sensor (102) to other medical conditions. For example, continuous monitoring of sodium, pH, lactate, oxygen, and glucose, in individuals experiencing diabetic ketoacidosis, can diagnose dehydration (loss of sodium), ketoacidosis (high glucose and low pH) and ischemia (low oxygen and high lactate), directing healthcare professionals towards a specific mode of intervention. An expanded flexible sensor (102) could also be applicable beyond the medical field, such as in bioreactors for protein expression, agriculture, water management, and food industry, where sensing pH, glucose, and sodium, would provide vital information to each respective field.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1-36. (canceled)

37. A multi-analyte monitoring (MAM) system comprising:
a) a multi-analyte sensor unit comprising:
i) a support substrate;
ii) a plurality of analyte sensors disposed on the support substrate, the plurality of analyte sensors configured to monitor a plurality of analytes, each analyte sensor comprising:
A) at least one opto-electronic component disposed on the support substrate;
B) a luminescent sheet comprising a porous sheet coated with a dye coating, wherein the luminescent sheet is disposed on the at least one opto-electronic component; and
b) a recording unit (103) operatively coupled to the sensor unit, wherein the recording unit (103) includes an analyte detector (303) for detecting luminescence from at least one of the analyte sensors.

38. The MAM system of claim 37, wherein the plurality of analyte sensors includes a pH sensor comprising:
a) at least one light source (301) disposed on the support substrate;
b) at least one photodiode (302) disposed on the support substrate, adjacent to the at least one light source (301); and
c) a pH sensor sheet (305) comprising a porous sheet coated with a polymer coating comprising particles and pH sensitive dye molecules bound thereto, wherein the pH sensor sheet is disposed on the at least one light source (301) and/or the at least one photodiode (302), wherein the at least one light source (301) is configured to illuminate the pH sensitive dye molecules, which produces a luminescence that is detectable by the at least one photodiode (302), wherein changes in the luminescence is related to changes in the pH.

39. The MAM system of claim 38, wherein the pH sensor further comprises a second light source (301) disposed on the support substrate, wherein the pH sensor sheet (305) is further disposed on the second light source (301), wherein the second light source (301) is configured to illuminate the pH sensitive dye molecules.

40. The MAM system of claim 39, wherein the second light source (301) is positioned such that the at least one photodiode (302) is disposed between the two light sources (301).

41. The MAM system of claim 38, wherein the at least one photodiode (302) is coated with an optical filter to pass a specific band or bands of wavelength.

42. The MAM system of claim 38, wherein the pH sensor further comprises a second photodiode (302) disposed on the support substrate, wherein the pH sensor sheet (305) is further disposed on the second photodiode (302), wherein the second photodiode (302) is configured to detect the luminescence of the pH sensitive dye molecules.

43. The MAM system of claim 42, wherein the second photodiode (302) is positioned such that the at least one light source (301) is disposed between the two photodiodes (302).

44. The MAM system of claim 42, wherein each photodiode (302) is coated with an optical filter to pass a specific band or bands of wavelength.

45. (canceled)

46. The MAM system of claim 38, wherein the recording unit (103) is configured to receive and record signals that correspond to the luminescence from the pH sensor.

47. (canceled)

48. The MAM system of claim 37, wherein the luminescent sheet comprises one or more catalysts configured to interact with a target during interaction with an analyte, and a luminescent dye configured to interact with the target and generate a luminescence corresponding to said analyte, wherein changes in said luminescence are related to the concentration of said analyte.

49. The MAM system of claim 48, wherein the one or more catalysts consume the target in the presence of the analyte and the luminescent dye, wherein the target binds or reacts to the luminescent dye and at least partially quenches the luminescence of the luminescent dye, thereby resulting in the change in luminescence.

50. (canceled)

51. The MAM system of claim 48, wherein the one or more analytes are lactate, glucose, ketones, or a combination thereof.

52. The MAM system of claim 48, wherein the one or more analyte sensors further comprise luminescent probes.

53. The MAM system of claim 52, wherein each luminescent probe comprises a fluorescent protein pair, wherein each fluorescent protein is conjugated to a macromolecule, wherein a binding event produces an energy transfer between the fluorescent protein pairs that yield luminescent signals.

54-61. (canceled)

62. The MAM system of claim 37, wherein the support substrate further comprises a flexible base layer and electrically conducting wires or traces (201) disposed on the flexible base layer and operatively coupling the recording unit (103) to the analyte sensors.

63-67. (canceled)

68. The MAM system of claim 37, wherein the opto-electronic components comprise light sources that can be activated one at a time to limit any optical signal crosstalk.

69-71. (canceled)

72. The MAM system of claim 37, wherein the analyte sensors are disposed on both sides of the supporting substrate.

73. The MAM system of claim 37, wherein the sensor unit is configured to be implanted under skin, in a tissue, or in an organ.

74. The MAM system of claim 37, wherein the recording unit (103) is configured to be placed on the skin or implanted under the skin, in the tissue, or in the organ such that the analyte detector (303) is positioned above or directly facing at least one of the analyte sensors of the sensor unit.

75-76. (canceled)

77. The MAM system of claim 52, wherein the luminescent probes are sensitive to peptides, sugars, lipids, hormones, nucleic acids, calcium, magnesium, zinc, and other ions or electrolytes.
